(19) United States
(12) Patent Application Publication
(10) Pub. No.: US 2004/0048780 A1
(43) Pub. Date: Mar. 11, 2004

(54) Method for treating and preventing cardiac arrhythmia

(75) Inventor: Andrew R. Marks, Larchmont, NY (US)

Correspondence Address: John P. White, Cooper & Dunham LLP, 1185 Avenue of the Americas, New York, NY, 10036, US

(73) Assignee: The Trustees of Columbia University in the City of New York

(21) Appl. No.: 10/608,723

(22) Filed: Jun. 26, 2003

Related U.S. Application Data
(63) Continuation-in-part of application No. 10/288,606, filed on Nov. 5, 2002.
Application No. 10/288,606 is a continuation of application No. 09/568,474, filed on May 10, 2000, now Pat. No. 6,489,125.

(51) Int. Cl. A61K 38/00

(52) U.S. Cl. 514/2

(57) ABSTRACT

This invention provides methods for treating a subject afflicted with atrial tachyarrhythmia and methods for inhibiting the onset of atrial tachyarrhythmia. This invention also provides methods for treating a subject afflicted with exercise-induced or stress-induced cardiac arrhythmia and methods for inhibiting the onset of exercise-induced or stress-induced cardiac arrhythmia.

This application is a continuation-in-part of U.S. Ser. No. 10/288,606, filed Nov. 5, 2002, which is a continuation of U.S. Ser. No. 09/568,474, filed May 10, 2000, now U.S. Pat. No. 6,489,125 B1, issued Dec. 3, 2002, the contents of which are incorporated herein by reference.

The invention disclosed herein was made with Government support under grant numbers RO1 HL61503 and RO1 HL56180 from the National Institutes of Health, U.S. Department of Health and Human Services. Accordingly, the U.S. Government has certain rights in this invention.

Throughout this application, various publications are referenced in parentheses by author and year. Full citations for these references may be found at the end of the specification immediately preceding the claims. The disclosures of these publications in their entireties are hereby incorporated by reference into this application to more fully describe the state of the art to which this invention pertains.

### BACKGROUND OF THE INVENTION

The contraction of striated muscle is initiated when calcium (Ca²⁺) is released from tubules within the muscle cell known as the sarcoplasmic reticulum (SR). Calcium release channels (ryanodine receptors, RyRs) on the SR of striated muscle are required for excitation-contraction (EC) coupling. The type 2 ryanodine receptor (RyR2) is the major Ca²⁺ release channel required for EC coupling in cardiac muscle. During EC coupling, depolarization of the cardiac muscle cell membrane in phase zero of the action potential activates voltage-gated Ca²⁺ channels. Ca²⁺ influx through these channels in turn initiates Ca²⁺ release via RyR2 from the SR in a process known as Ca²⁺-induced Ca²⁺ release (Fabiato, 1983; Nabauer et al., 1989). RyR2-mediated, Ca²⁺-induced Ca²⁺ release activates the contractile proteins, which are responsible for cardiac muscle contraction.

The RyR2 receptor is a tetramer comprising four 565,000 dalton RyR2 polypeptides and four 12,000 dalton FK-506 binding proteins (FKBP12.6). FKBP12.6 binds to the RyR2 channel, one molecule per RyR2 subunit, stabilizes RyR2 channel function (Brillantes et al., 1994) and facilitates coupled gating between neighboring RyR2 channels (Marx et al., 1998). The latter are packed into dense arrays in specialized regions of the SR that release intracellular stores of Ca²⁺, triggering muscle contraction. A homologous type 1 ryanodine receptor (RyR1) which binds a regulatory subunit, FKBP12, is found on the SR of skeletal muscle.

It has been previously shown that in failing hearts, hyperphosphorylation of RyR2 by cAMP-dependent protein kinase A (PKA) induces the dissociation of the regulatory FKBP12.6 subunit from the RyR2 channel (Marx et al., 2000). This causes marked changes in the biophysical properties of the RyR2 channel, evidenced by increased open probability (Pₒ) due to an increased sensitivity to Ca²⁺-dependent activation (Brillantes et al., 1994; Kaftan et al., 1996), destabilization of the channel resulting in subconductance states, and impaired coupled gating of the channels, resulting in defective EC coupling and cardiac dysfunction (Marx et al., 1998).

Another common feature of heart failure is the occurrence of cardiac arrhythmias. Ventricular arrhythmias in the heart can be rapidly fatal, a phenomenon referred to as sudden cardiac death (SCD). SCD is associated with common cardiac diseases, most notably heart failure, in which approximately 50% of patients die from fatal cardiac arrhythmias. However, fatal ventricular arrhythmias can also occur in young, otherwise healthy individuals without known structural heart disease. In structurally normal hearts the most common mechanism for induction and maintenance of ventricular tachycardia is abnormal automaticity. One form of abnormal automaticity, known as “triggered arrhythmias,” is associated with aberrant release of SR Ca²⁺ that initiates delayed after-depolarizations (DADs) (Fozzard, 1992; Wit and Rosen, 1983). DADs, which can trigger fatal ventricular arrhythmias, are abnormal depolarizations in cardiomyocytes that occur after repolarization of a cardiac action potential. The molecular basis for abnormal SR Ca²⁺ release that causes DADs has not been fully elucidated. DADs are, however, known to be blocked by ryanodine, providing evidence that RyR2 may play a key role in the pathogenesis of this aberrant Ca²⁺ release (Marban et al., 1986; Song and Belardinelli, 1994).

In subjects with an inherited, arrhythmogenic disorder known as catecholaminergic polymorphic ventricular tachycardia (CPVT), physical exertion and emotional stress induce bidirectional and/or polymorphic ventricular tachycardias that lead to SCD in the absence of detectable structural heart disease (Laitinen et al., 2001; Leenhardt et al., 1995; Priori et al., 2002; Priori et al., 2001; Swan et al., 1999). CPVT is predominantly inherited in an autosomal-dominant fashion. Affected individuals present during childhood or adolescence with repetitive exercise-induced syncopal events with 30-50% mortality by age 30 (Fisher et al., 1999; Swan et al., 1999). Linkage studies and direct sequencing have identified mutations in the human RyR2 gene (hRyR2) on chromosome 1q42-q43 in individuals with CPVT (Laitinen et al., 2001; Priori et al., 2001; Swan et al., 1999). Importantly, individuals with CPVT have ventricular arrhythmias when subjected to exercise testing, but they do not have these arrhythmias at rest.

Stimulation of the sympathetic nervous system during exercise and emotional stress is known to cause the release of catecholamines that activate β-adrenergic receptors (β-ARs) in the heart. β-ARs are coupled via G-proteins to the activation of adenylyl cyclase that increases intracellular cAMP concentration that in turn activates PKA.

### SUMMARY OF THE INVENTION

The present invention provides a method for treating a subject afflicted with atrial tachyarrhythmia comprising administering to the subject a therapeutically effective amount of an agent which inhibits protein kinase A (PKA) phosphorylation of a type 2 ryanodine receptor (RyR2) in the subject's heart, thereby treating the subject.

This invention also provides a method for treating a subject afflicted with atrial tachyarrhythmia comprising administering to the subject a therapeutically effective amount of an agent which inhibits dissociation of a FKBP12.6 binding protein from a type 2 ryanodine (RyR2) receptor in the subject's heart, thereby treating the subject.

This invention further provides a method for treating a subject afflicted with atrial tachyarrhythmia comprising administering to the subject a therapeutically effective amount of an agent which mimics binding of a FKBP12.6 binding protein to a type 2 ryanodine receptor (RyR2) of the subject's heart, thereby treating the subject.

In addition, this invention is directed to an article of manufacture comprising (i) a packaging material having therein an agent which inhibits protein kinase A (PKA) phosphorylation of a type 2 ryanodine receptor (RyR2) and (ii) a label indicating a use for the agent in treating a subject afflicted with atrial tachyarrhythmia.

This invention is also directed to an article of manufacture comprising (i) a packaging material having therein an agent which inhibits dissociation of a FKBP12.6 binding protein from a type 2 ryanodine receptor (RyR2) and (ii) a label indicating a use for the agent in treating a subject afflicted with atrial tachyarrhythmia.

This invention is further directed to an article of manufacture comprising (i) a packaging material having therein an agent which mimics binding of a FKBP12.6 binding protein to a type 2 ryanodine receptor (RyR2) and (ii) a label indicating a use for the agent in treating a subject afflicted with atrial tachyarrhythmia.

This invention additionally provides a method for inhibiting the onset of atrial tachyarrhythmia in a subject comprising administering to the subject a prophylactically effective amount of an agent which inhibits protein kinase A (PKA) phosphorylation of a type 2 ryanodine receptor (RyR2) in the subject's heart, thereby inhibiting the onset of an atrial tachyarrhythmia in the subject.

This invention also provides a method for inhibiting the onset of atrial tachyarrhythmia in a subject comprising administering to the subject a prophylactically effective amount of an agent which inhibits dissociation of a FKBP12.6 binding protein from a type 2 ryanodine (RyR2) receptor in the subject's heart, thereby inhibiting the onset of atrial tachyarrhythmia in the subject.

This invention further provides a method for inhibiting the onset of atrial tachyarrhythmia in a subject comprising administering to the subject a prophylactically effective amount of an agent which mimics binding of a FKBP12.6 binding protein to a type 2 ryanodine receptor (RyR2) of the subject's heart, thereby inhibiting the onset of atrial tachyarrhythmia in the subject.

In addition, this invention is directed to an article of manufacture comprising (i) a packaging material having therein an agent which inhibits protein kinase A (PKA) phosphorylation of a type 2 ryanodine receptor (RyR2) and (ii) a label indicating a use for the agent in inhibiting the onset of atrial tachyarrhythmia in a subject.

This invention is also directed to an article of manufacture comprising (i) a packaging material having therein an agent which inhibits dissociation of a FKBP12.6 binding protein from a type 2 ryanodine receptor (RyR2) and (ii) a label indicating a use for the agent in inhibiting the onset of atrial tachyarrhythmia in a subject.

This invention is further directed to an article of manufacture comprising (i) a packaging material having therein an agent which mimics binding of a FKBP12.6 binding protein to a type 2 ryanodine receptor (RyR2) and (ii) a label indicating a use for the agent in inhibiting the onset of atrial tachyarrhythmia in a subject.

The present invention provides a method for treating a subject afflicted with exercise-induced or stress-induced cardiac arrhythmia comprising administering to the subject a therapeutically effective amount of an agent which inhibits protein kinase A (PKA) phosphorylation of a type 2 ryanodine receptor (RyR2) in the subject's heart, thereby treating the subject.

This invention also provides a method for treating a subject afflicted with exercise-induced or stress-induced cardiac arrhythmia comprising administering to the subject a therapeutically effective amount of an agent which inhibits dissociation of a FKBP12.6 binding protein from a type 2 ryanodine (RyR2) receptor in the subject's heart, thereby treating the subject.

This invention further provides a method for treating a subject afflicted with exercise-induced or stress-induced cardiac arrhythmia comprising administering to the subject a therapeutically effective amount of an agent which mimics binding of a FKBP12.6 binding protein to a type 2 ryanodine receptor (RyR2) of the subject's heart, thereby treating the subject.

In addition, this invention provides an article of manufacture comprising (i) a packaging material having therein an agent which inhibits protein kinase A (PKA) phosphorylation of a type 2 ryanodine receptor (RyR2) and (ii) a label indicating a use for the agent in treating a subject afflicted with exercise-induced or stress-induced cardiac arrhythmia.

This invention also provides an article of manufacture comprising (i) a packaging material having therein an agent which inhibits dissociation of a FKBP12.6 binding protein from a type 2 ryanodine receptor (RyR2) and (ii) a label indicating a use for the agent in treating a subject afflicted with exercise-induced or stress-induced cardiac arrhythmia.

This invention further provides an article of manufacture comprising (i) a packaging material having therein an agent which mimics binding of a FKBP12.6 binding protein to a type 2 ryanodine receptor (RyR2) and (ii) a label indicating a use for the agent in treating a subject afflicted with exercise-induced or stress-induced cardiac arrhythmia.

The present invention additionally provides a method for inhibiting the onset of exercise-induced or stress-induced cardiac arrhythmia in a subject comprising administering to the subject a prophylactically effective amount of an agent which inhibits protein kinase A (PKA) phosphorylation of a type 2 ryanodine receptor (RyR2) in the subject's heart, thereby inhibiting the onset of exercise-induced or stress-induced cardiac arrhythmia in the subject.

This invention also provides a method for inhibiting the onset of exercise-induced or stress-induced cardiac arrhythmia comprising administering to the subject a prophylactically effective amount of an agent which inhibits dissociation of a FKBP12.6 binding protein from a type 2 ryanodine (RyR2) receptor in the subject's heart, thereby inhibiting the onset of exercise-induced or stress-induced cardiac arrhythmia in the subject.

This invention further provides a method for inhibiting the onset of exercise-induced or stress-induced cardiac arrhythmia comprising administering to the subject a prophylactically effective amount of an agent which mimics binding of a FKBP12.6 binding protein to a type 2 ryanodine receptor (RyR2) of the subject's heart, thereby inhibiting the onset of exercise-induced or stress-induced cardiac arrhythmia in the subject.

In addition, the present method provides an article of manufacture comprising (i) a packaging material having therein an agent which inhibits protein kinase A (PKA) phosphorylation of a type 2 ryanodine receptor (RyR2) and (ii) a label indicating a use for the agent in inhibiting the onset of exercise-induced or stress-induced cardiac arrhythmia in a subject.

This invention also provides an article of manufacture comprising (i) a packaging material having therein an agent which inhibits dissociation of a FKBP12.6 binding protein from a type 2 ryanodine receptor (RyR2) and (ii) a label indicating a use for the agent in inhibiting the onset of exercise-induced or stress-induced cardiac arrhythmia in a subject.

Finally, this invention provides an article of manufacture comprising (i) a packaging material having therein an agent which mimics binding of a FKBP12.6 binding protein to a type 2 ryanodine receptor (RyR2) and (ii) a label indicating a use for the agent in inhibiting the onset of exercise-induced or stress-induced cardiac arrhythmia in a subject.

### BRIEF DESCRIPTION OF THE FIGURES

FIG. 1. PKA phosphorylation of RyR2, a macromolecular signaling complex. (A) RyR2 was phosphorylated by addition of PKA (5 units) or 3,5′-cyclic adenosine 5′-monophosphate (cAMP) (10 μM); the PKA inhibitor PKI₅₋₂₄ (500 nM) inhibited the phosphorylation. RyR2 was immunoprecipitated and subjected to in vitro kinasing reactions; equivalent amounts of RyR2 protein were used in each reaction as shown by immunoblotting. (B) RyR2 channels were isolated using [³H] ryanodine by centrifugation on a sucrose density gradient as described by Brillantes et al. (1994). Both [3H] ryanodine binding (open squares) and total protein (filled circles) were plotted. Individual RyR2 channels sediment at 30S (arrow), and two or more physically attached RyR2 channels sediment in higher sucrose fractions as previously reported for RyR1 channels (Marx et al., 1998). (C) Immunoblotting gradient fractions with specific antibodies showed that FKBP12.6, PKA catalytic subunit (PKA cat), PKA regulatory subunit (RII), protein phosphatase 2A (PP2A), protein phosphatase 1 (PP1), muscle A-kinase anchoring protein (mAKAP), but not calcineurin (CnA), were detected in all fractions containing RyR2. (D) RyR2 binding to microcystin-Sepharose beads was competed using free microcystin-LR. Samples were pelleted and analyzed by SDS-PAGE and immunoblotting with anti-RyR antibody. (E) Components of the RyR2 complex (FKBP12.6, PKA, RII, PP2A, PP1, and mAKAP) were co-immunoprecipitated from cardiac SR (200 μg protein). The RyR2 complex was sedimented using microcystin-Sepharose, the complex was competed off with free microcystin-LR followed by immunoprecipitation with anti-RyR antibody (αRyR) and immunoblotting. Positive controls (+Cont.) were recombinant or purified proteins as indicated; negative controls (−Cont.) were blocking peptides for each antibody or pre-absorbing the antibodies with purified or recombinant proteins. In all cases data shown are representative of more than three similar experiments.

FIG. 2. RyR2 PKA phosphorylation during heart failure. (A) PKA back-phosphorylation of RyR2 protein immunoprecipitated from the indicated tissues are shown in the top row; the middle row shows the amount of RyR2 immunoprecipitated in each reaction; and the bottom row shows the amount of PKA co-immunoprecipitated with RyR2 from each sample. Normal, non-failing human heart; ICM, end-stage failing human heart with ischemic cardiomyopathy; IDCM, end-stage failing human heart with idiopathic dilated cardiomyopathy; IDCM(-Dba) samples from patients not treated with a β-adrenergic agonist Dba (dobutamine); Pre-LVAD, left ventricular sample taken from a human heart with end-stage failure during insertion of a left ventricular assist device (LVAD); Post-LVAD, sample from the same human heart after LVAD treatment; PKI, representative negative control showing that PKA phosphorylation was inhibited by PKI. (B) Quantitation of RyR2 back-phosphorylation studies shown in (A). The inset shows PKA hyperphosphorylation of RyR2 confirmed by anti-phosphoserine immunoblotting: 1) top row, RyR2 immunoblot (top lanes); 2) bottom row, anti-phosphoserine immunoblotting of the same samples. Lane 1, normal non-failing human heart; lane 2, failing human (ICM) heart. For each condition a minimum of three experiments using tissue from three different hearts were performed, error bars represent standard deviation of the mean. (C) PP1 and PP2A co-immunoprecipitated with RyR2 from normal and failing hearts. Following immunoprecipitation with anti-RyR antibody, immunoprecipitates were size fractionated and immunoblotted with: anti-RyR2 (top panel), anti-PP1 (middle panel), or anti-PP2A antibodies. Data shown are representative of three similar experiments. (D) The amount of PP1 and PP2A co-immunoprecipitating with RyR2 was determined by densitometric quantitation of the immunoblots and normalized for the amount of RyR2 co-immunoprecipitated. Less PP1 and PP2A were associated with RyR2 in all of the heart failure samples. Data shown are representative of three similar experiments.

FIG. 3. Mapping signaling complex binding sites on RyR2. (A) The FKBP12.6 binding site in RyR2 was identified using a yeast two-hybrid interaction screen. The left bar-graph shows β-galactosidase activity for yeast transformed with: 1) FKBP12.6/activation domain fusion protein alone; 2) RyR2 (residues 2361-2496)/DNA binding domain alone; 3) both together. Interaction between FKBP12.6 and an RyR2 fragment (residues 2361-2496, Otsu et al., 1990) activates Gal-4 transcription resulting in increased β-galactosidase activity. The bar-graph shows normalized β-galactosidase activity for rapamycin-resistant yeast transformed with FKBP12.6 and the RyR2 fragment treated with the indicated concentrations of rapamycin which competes FKBP12.6 off from RyR2. The FKBP12/12.6 binding site in RyR2 is defined by isoleucine 2427 and proline 2428 (arrow). Shown in the box are sequences of FKBP12 binding sites in RyR1 (SEQ ID NO: 1) (Takeshima et al., 1989), RyR2 (SEQ ID NO: 2) (Otsu et al., 1990), IP3R1 (SEQ ID NO: 3) (Harnick et al., 1995), IP3R2 (SEQ ID NO: 4) (Yamamoto-Hino et al., 1994), and TβRI (SEQ ID NO: 5) (Franzen et al., 1993). (B) Glutathione-S-Transferase (GST)-RyR2 fusion proteins bound to Sepharose beads were incubated with cardiac SR (200 μg protein), pelleted, size fractionated by SDS-PAGE and immunoblotted with the indicated antibodies. Lane 1, positive control (recombinant proteins); lane 2, Sepharose beads (negative control); lane 3, GST (negative control); lane 4, GST-RyR2-1-334 (amino acid residues 1-334); lane 5, GST-RyR2-513-808; lane 6, GST-RyR2-1027-1304; lane 7, GST-RyR2-1251-1500; lane 8, GST-RyR2-1451-1768. (C) Immunohistochemistry showing co-localization of mAKAP and RyR2 to cardiac SR in normal and failing human hearts. Bars: long, 1.5 μm; short 5 μm. (D) In vitro kinasing reactions using GST-RyR2 fusion proteins containing the wild type (WT) and mutant (S2809A) PKA site. PKA phosphorylation was performed with [γ³²P]-ATP followed by size fractionation on SDS-PAGE and autoradiography.

FIG. 4. PKA phosphorylation of RyR2 inhibits FKBP12.6 binding. (A) FKBP12.6 was co-immunoprecipitated from cardiac SR using an anti-RyR antibody followed by immunoblotting with either anti-RyR (top panel) or anti-FKBP (bottom panel) antibodies. The indicated immunoprecipitates were phosphorylated with PKA prior to size fractionation by SDS-PAGE. Co-immunoprecipitation of FKBP12.6 with RyR2 was significantly reduced in the PKA phosphorylated samples but not in Ca²⁺-calmodulin kinase (CaMKII) or protein kinase C (PKC) phosphorylated RyR2 samples. RyR2 phosphorylation, as assessed using [γ³²P]-ATP, was equivalent for PKA, CamKII and PKC (not shown). (B) Quantitation of the amount of FKBP12.6 co-immunoprecipitating with RyR2 from the indicated samples. Normal human heart; pre-LVAD, left ventricular sample taken from a human heart with end-stage failure during insertion of a left ventricular assist device; Post-LVAD, sample from the same human heart after LVAD treatment; IDCM, end-stage failing human heart with idiopathic dilated cardiomyopathy; normal non-failing canine heart; canine rapid pacing-induced heart failure model. Inset shows representative co-immunoprecipitations of RyR2 and FKBP12.6 using an anti-RyR antibody: lane 1, normal human heart; lane 2, pre-LVAD; lane 3, post-LVAD; lane 4, human IDCM; lane 5, normal canine heart; lane 6, failing canine heart. There was significantly less FKBP12.6 co-immunoprecipitated with RyR2 in each of the failing hearts compared to normals. Data shown are representative of three or more similar experiments. FKBP12.6 amounts were quantified using densitometry of the specific FKBP12.6 band on a single immunoblot for each experiment.

FIG. 5. PKA phosphorylation activates RyR2 and induces subconductance states. (A) Open probability (Pₒ) of a single RyR2 channel plotted as a function of time showing the effect of MgATP (2 mM) followed by addition of PKA (2 units). (B) Single channel tracings corresponding to the experiment shown in A. Channel openings are in the upward direction, the current amplitude for a fully open channel under these conditions (Ba²⁺ as current carrier) was ˜4 pA. Increased Pₒ and multiple subconductance states are seen after PKA addition. Corresponding amplitude histograms are shown at the right of the tracings for channels before and after PKA treatment. The subconductance states have current amplitudes of 1, 2, or 3 pA, corresponding to ¼, ½ and ¾ of the full conductance of the channel as previously described for channels in the absence of FKBP12 (Brillantes et al., 1994; Marx et al., 1998). The bottom tracing shows the characteristic modification of the RyR2 channels by ryanodine (1 μM) which locks the PKA phosphorylated RyR2 channel in a one-half conductance state. Recordings were at 0 mV potential across the lipid bilayer membrane; the dashed lines indicate the closed state of the channels. Data shown are representative of 4 experiments using SR microsomes containing RyR2 isolated from 2 different dogs (2 separate isolations for each animal). Similar results were obtained using RyR2 channels isolated from normal human heart.

FIG. 6. Defective RyR2 channels and contractility in failing heart muscle. (A) Single channel tracings of RyR2 from: normal canine heart (top three tracings); failing canine heart (bottom three tracings). Corresponding amplitude histograms are at right. The bottom tracing in each set of three shows the characteristic modification of the RyR2 channels by ryanodine (1 μM) which locks the channel in a ½ conductance state. Recordings were at 0 mV; the dashed lines indicate the closed state of the channels. Similar results were obtained using RyR2 channels isolated from failing human hearts (see text for details) (B) RyR2 channels from failing canine hearts exhibited increased sensitivity to Ca²⁺-dependent activation compared to channels from normal hearts which were generally inactive at ≦50 nM free Ca²⁺ in the cis (cytoplasmic) chamber (top tracing). RyR2 channels from failing hearts exhibited two types of Ca²⁺-dependent activation at ≦50 nM free Ca²⁺. Some channels from failing hearts (n=15) were active with a low Pₒ (second tracing) at ≦50 nM free Ca²⁺; others (n=4) were extremely active at ≦50 nM free Ca²⁺ remaining stably open in a subconductance state (bottom tracing). Similar results were obtained using RyR2 channels isolated from failing human hearts. (C) Continuous force tracings from human left ventricular trabeculae during exposure to isoproterenol (4 μM). Normal heart sample showed >3-fold increase in force following isoproterenol. Muscles from patients with congestive heart failure (CHF) showed either no response in patients receiving β-agonists prior to transplant, or a blunted response (˜2-fold increase) in patients not receiving β-agonists prior to transplant. Muscle obtained from the apical core tissue of an LVAD recipient receiving β-agonist prior to surgery showed almost no response to isoproterenol. However, muscle obtained from the same patient after 64 days of LVAD support shows a >5-fold increase in response to isoproterenol (n=3, p<0.01).

FIG. 7. Model of the effects of PKA phosphorylation of RyR2 in the heart. In the non-failing heart (left panel) β-agonists bind to receptors (β1 and β2 adrenergic receptors, ARs) coupled to heterotrimeric G-proteins (Gprot) which in turn activate adenylyl cyclase (AC) raising cyclic AMP (cAMP) levels and activating PKA. In this model PKA phosphorylation of RyR2 induces dissociation of one FKBP12.6 from the channel shifting the Ca²⁺-dependence for activation to the left, increasing the sensitivity of the RyR2 to activation by Ca²⁺ influx via the voltage-gated calcium channel in the T-tubule (dihydropyridine receptor, DHPR) and increasing RyR2 channel open probability. The result is increased SR Ca²⁺ release and cardiac contractility. The tetrameric RyR2 channel is part of a macromolecular signaling complex that includes four molecules each of RyR2, FKBP12.6, PKA, protein phosphatases PP1 and PP2A, and the anchoring protein mAKAP (the PKA, PP1, PP2A, mAKAP components of the macromolecular complex are shown for only one of the four RyR2 subunits). Ca²⁺ reuptake into the SR occurs via the SR Ca²⁺-ATPase (SERCA) and its associated regulatory protein phospholamban (PLB).

In failing hearts (right panel) PKA hyperphosphorylation of RyR2 may contribute to the blunted response to β-agonists observed in failing heart muscle because the channels cannot be further PKA phosphorylated. RyR2 channels in failing hearts exhibit a shift in the Ca²⁺-dependence for activation such that they are activated at resting levels of cytosolic Ca²⁺.

FIG. 8. Generation of FKBP12.6-deficient mice. (A) Restriction maps of the mouse FKBP12.6 gene (top), targeting vector (middle), and homologous recombinant mutant allele (bottom). A 3.5 kilobase (kb) SpeI-SacI segment that includes exons 3 and 4 was replaced with a neomycin-resistance (neo) expression cassette. A thymidine kinase (TK) cassette was linked to the 3′ end for purposes of negative selection. (B) Southern blot analysis of DNA extracted from tails of mice heterozygous (+/−) or homozygous (−/−) for the FKBP12.6 allele, and wild type mouse (+/+). DNA was digested with BglII, and hybridized with a 5′ probe (indicated in panel A), yielding an 8.5 kb and 7.7 kb band for wild type (+/+) and FKBP12.6-deficient (−/−) mice, respectively. (C) FKBP12/12.6 immunoblot of heart homogenate from FKBP12.6 (+/+) and (−/−) mice.

FIG. 9. Exercise-induced fatal ventricular arrhythmias and delayed after-depolarizations in FKBP12.6-deficient mice. There were no significant abnormalities comparing the ECGs of (A) conscious FKBP12.6^{+/+} mice to (B) FKBP12.6^{-/-} mice. (C) ECG of FKBP12.6^{-/-} mouse following exercise and epinephrine. Strenuous exercise and injection with epinephrine (˜8 minutes before the trace) induces polymorphic ventricular tachycardia in a FKBP12.6^{-/-} mouse, followed by death 4 minutes after this trace was recorded. This stress test resulted in ventricular arrhythmias in 8/8 (100%) of male (n=3) and female (n=5) FKBP12.6^{-/-} mice, compared to 0/6 (0%) of the FKBP12.6^{+/+} mice. Action potentials were recorded in (D) FKBP12.6^{+/+} (n=19) and (E) FKBP12.6^{-/-} (n=38) cardiomyocytes during rapid pacing. Action potentials were produced in patch-clamped single cardiomyocytes stimulated by brief current injections at 12 Hz in current clamp mode. Upward arrows at the bottom of panels (D) and (E) indicate the timing of the induced action potentials. After-depolarizations such as the one shown in (E) were observed in FKBP12.6^{-/-} cardiomyocytes (n=6) from four FKBP12.6^{-/-} mice. The downward arrow shows a delayed after-depolarization (DAD) that produced an extrasystole in a FKBP12.6^{-/-} cardiomyocyte.

FIG. 10. Single channel studies showing increased open probability of RyR2 from FKBP12.6-deficient mice during exercise. (A) PKA phosphorylation of RyR2 from FKBP12.6^{+/+} and FKBP12.6^{-/-} mice is increased during exercise. Mice were subjected to exercise as described in “Materials and Methods,” and at the termination of exercise the heart was rapidly removed and flash frozen. RyR2 was immunoprecipitated from cardiac homogenates and PKA phosphorylation of RyR2 was assessed using a phosphoepitope-specific antibody (anti-RyR2-P2809) that specifically recognizes RyR2 PKA-phosphorylated at RyR2-Serine²⁸⁰⁹. Immunoblots of RyR2 with anti-RyR2-5029 (Jayaraman et al., 1992), an antibody that recognizes the carboxy terminus of RyR2 were used to show that equal amounts of RyR2 protein were examined in each sample. (B) The amount of FKBP12.6 bound to RyR2 after exercise was decreased (due to PKA phosphorylation of RyR2 which dissociates FKBP12.6 from the channel) in FKBP12.6^{+/+} mice. No FKBP12.6 was detected in the RyR2 macromolecular complex in FKBP12.6^{-/-} mice. (C)-(F) Single channel studies showing increased open probability of RyR2 from FKBP12.6^{-/-} mice compared to FKBP12.6^{+/+} after exercise. For each condition, channel openings are upward, the dash indicates the level of full openings (4 pA), and ‘c’ indicates the closed state. Channels are shown at compressed (5 sec, upper tracing) and expanded (500 ms, lower tracing) time scales; recordings are at 0 mV. Amplitude histograms of FKBP12.6^{+/+} and FKBP12.6^{-/-} channels reveal increased activity, and partial or subconductance openings in exercised FKBP12.6^{-/-} channels. (G) Summary of open probabilities (Pₒ) of control and exercised FKBP12.6^{+/+} and FKBP12.6^{-/-} mice, revealing increased Pₒ in FKBP12.6^{-/-} channels after exercise (note interruption in y-axis) under conditions (low cytosolic [Ca²⁺]=150 nM) that simulate diastole (the resting phase) in the heart.

FIG. 11. RyR2 mutations linked to exercise-induced sudden cardiac death reduce FKBP12.6 affinity and increase RyR2 activation by PKA phosphorylation. (A) Location of CPVT-associated RyR2 mutations. Known CPVT mutations in RyR2 are indicated with thin vertical lines; the three CPVT mutations analyzed in this application are indicated with thick vertical lines, and labeled with corresponding amino acid substitutions. ‘FKBP12.6’ binding site is shown on RyR2; ‘S2809’ is the PKA phosphorylation site on RyR2. Three regions containing clusters of skeletal muscle RyR1 mutations linked to malignant hyperthermia (MH) and central core disease (CCD) are indicated with thick grey lines. CPVT and MH/CCD mutations occur in highly homologous regions of RyR2 and RyR1, respectively, suggesting that common defects in SR Ca²⁺ release may play a role in these disorders. (B) CPVT-mutant RyR2 channels exhibit altered channel activity only under conditions that simulate exercise. Single channel recordings of WT and CPVT-mutant RyR2 expressed in HEK293 cells treated with PKA plus the PKA inhibitor PKI₅₋₂₄. RyR2-WT and CPVT-associated mutant channels exhibit low activity under basal conditions with cis (cytosolic) [Ca²⁺] of 150 nM. PKA-phosphorylated CPVT-mutant RyR2 exhibit increased activity compared to RyR2-WT channels. A minimum of 5 channels from at least two separate preparations were recorded for the RyR2-WT and each mutant RyR2 channel for each condition shown. (C) Average open probabilities (Pₒ) of RyR2-WT and CPVT-associated mutant RyR2 channels after PKA phosphorylation; * indicates P<0.05. (D) ³⁵S-labeled FKBP12.6 binding to microsomes containing RyR2-WT and CPVT-associated mutant RyR2 channels indicating decreased FKBP12.6 affinity for the CPVT-associated mutant RyR2 channels. (E) RyR2-WT and one of the CPVT-associated mutant RyR2 (RyR2-R2474S) were expressed as homo- or heterotetrameric channels. Representative single channel traces of RyR2-WT, RyR2-R2474S, and heterotetrameric WT×R2474S channels are shown, revealing increased activity of WT×R2474S channels after PKA phosphorylation. (F) Bar graph summarizing average open probabilities for RyR2-WT, WT×R2474S, and RyR2-R2474S channels; * indicates P<0.05.

FIG. 12. A mutant FKBP12.6-D37S restores normal single channel function to channels from exercised FKBP12.6^{-/-} mice and PKA-phosphorylated CPVT-mutant channels. (A) Immunoblot showing equivalent amounts of RyR2 immunoprecipitated from murine cardiac SR(CSR), and recombinant RyR2-S2809D expressed in HEK293 cells, using anti-RyR-5029 antibody that recognizes the carboxy terminus of RyR (Jayaraman et al., 1992). Immunoblot of FKBP12.6 showing dissociation of FKBP12.6 from RyR2 following PKA phosphorylation. Wild-type FKBP12.6 does not bind to PKA-phosphorylated RyR2. The mutant FKBP12.6-D37S bound to PKA-phosphorylated RyR2 channels. RyR2-S2809D mimics constitutively PKA-phosphorylated RyR2 channels, and therefore does not bind FKBP12.6. However, mutant FKBP12.6-D37S binds RyR2-S2809D channels. (B) Single channel tracings of RyR2-S2809D channels treated with FKBP12.6 (top) and mutant FKBP12.6-D37S (bottom), showing increased open probability of RyR2-S2809D channels, similar to PKA-phosphorylated RyR2 channels (Marx et al., 2000). Binding of FKBP12.6-D37S to RyR2-S2809D channels rescued normal channel gating and decreased open probability (bottom). Corresponding amplitude histograms are shown on the right, revealing the presence of partial openings or subconductance states in the FKBP12.6-treated channels, but not in the presence of FKBP12.6-D37S. (C) Immunoblots of RyR2 and FKBP12.6 of CSR from exercised male (M) and female (F) FKBP12.6^{-/-} mice. Whereas wild-type FKBP12.6 does not, FKBP12.6-D37S does bind to RyR2 isolated from exercised FKBP12.6^{-/-} mouse hearts. (D) Single channel tracings of RyR2 channels isolated from exercised FKBP12.6^{-/-} hearts, showing high open probability and subconductance states (see histogram on the right). Binding of FKBP12.6-D37S completely rescued the channel phenotype: open probability is dramatically decreased, and no subconductance openings were observed. (E) Immunoblots of recombinant RyR2-R2474S expressed in HEK293 cells. FKBP12.6-D37S also binds to PKA-phosphorylated, CPVT-associated mutant RyR2-R2474S channels. (F) Single channel tracings of CPVT-associated mutant RyR2-R2474S channels after PKA phosphorylation, revealing high open probability and the presence of subconductance states (see amplitude histogram). Binding of FKBP12.6-D37S completely normalized channel gating and open probability. For each tracing, channel openings are upward, the dash indicates the level of full openings (4 pA), and ‘c’ indicates the closed state. Channels are shown at compressed (5 sec, upper tracing) and expanded (500 ms, lower tracing) time scales; recordings are at 0 mV.

FIG. 13. Model of mechanism by which SR Ca²⁺ leak may initiate delayed after-depolarizations that trigger cardiac arrhythmias. During excitation-contraction (EC) coupling in cardiac muscle, the cardiac ryanodine receptor (RyR2) on the sarcoplasmic reticulum (SR) is activated by the influx of Ca²⁺ through the voltage-gated calcium channel (VGCC) on the transverse tubule (T-tubule). In response to stress/exercise-induced activation of the sympathetic nervous system, RyR2 is PKA-phosphorylated which causes dissociation of FKBP12.6 from the channel and increases its open probability. This is part of a normal physiological stress response (e.g., “fight or flight”). In normal hearts the wild-type RyR2 is able to remain tightly shut, even when it is physiologically PKA-phosphorylated, during diastole when the heart muscle is relaxed so that it can re-fill with blood in preparation for the next contraction (systole). This is necessary to prevent SR Ca²⁺ leak during diastole which can initiate delayed after-depolarizations (DADs) that can trigger cardiac arrhythmias (Fozzard, 1992; Wit and Rosen, 1983). In the case of the CPVT-associated RyR2 mutant channels, increased activity (increased open probability) of the channels when they are PKA-phosphorylated (due to decreased binding affinity of FKBP12.6) increases the probability of aberrant SR Ca²⁺ leak during diastole which can activate DADs due to inward depolarizing currents, possibly through the sodium/calcium exchanger, that are triggers for cardiac arrhythmias (Pogwizd et al., 1998). Similar mechanisms may underlie cardiac arrhythmias in heart failure in which structurally normal RyR2 are PKA-hyperphosphorylated resulting in defective channel function (Marx et al., 2000) that may promote SR Ca²⁺ leak that can trigger arrhythmias (Pogwizd et al., 2001). The likelihood of cardiac arrhythmias triggered by activation of inward depolarizing currents via the sodium/calcium exchanger is further enhanced in heart failure due to upregulation of the exchanger (Pogwizd et al., 2001).

### DETAILED DESCRIPTION OF THE INVENTION

Definitions

The following definitions are presented as an aid in understanding this invention.

As used herein a “RyR2 receptor” means a type 2 ryanodine receptor, which is a calcium (Ca²⁺) release channel on the sarcoplasmic reticulum (SR) of the heart.

“FKBP12.6” means a FK-506 binding protein, having a molecular weight of about 12,000 daltons, that binds to and regulates the gating (activation and inactivation) of the RyR2 receptor channel.

“PKA phosphorylation” means the substitution of a hydroxyl group in a substrate by a phosphate group in a reaction catalyzed by the enzyme, cAMP-dependent protein kinase A (PKA).

“Back-phosphorylation” of the RyR2 receptor means the in vitro phosphorylation of RyR2 by PKA.

“Catecholaminergic polymorphic ventricular tachycardia”, also referred to as “CPVT”, is a disorder characterized by adrenergic- (and thus exercise- and mental stress-) induced bi-directional and/or polymorphic ventricular tachycardias and sudden cardiac death in the absence of gross structural disease of the myocardium. A “ventricular tachycardia” is an abnormally rapid heart rhythm associated with the generation of electrical impulses within the ventricles (at least three consecutive ventricular complexes of more than 100 beats per minute), and is characterized by an electrocardiogram having a broad QRS complex.

A “pharmaceutically effective amount” is any amount of an agent which, when administered to a subject suffering from a disorder against which the agent is effective, causes reduction, remission or regression or prevents recurrence of the disorder.

A “prophylactically effective amount” is any amount of an agent which, when administered to a subject prone to suffer from a disorder, inhibits the onset of the disorder.

“Inhibiting” the onset of a disorder shall mean either lessening the likelihood of the disorder's onset, or preventing the onset of the disorder entirely. In the preferred embodiment, inhibiting the onset of a disorder means preventing its onset entirely. Determining a therapeutically or prophylactically effective amount of the instant agents can be done based on animal data using routine computational methods. In one embodiment, the therapeutically or prophylactically effective amount contains between about 0.2 mg and about 1.5 g of the agent. In another embodiment, the effective amount contains between about 5 mg and about 500 mg of the agent. In a further embodiment, the effective amount contains between about 25 mg and about 125 mg of the agent.

“Pharmaceutically acceptable carriers” are well known to those skilled in the art and include, but are not limited to, 0.01-0.1 M and preferably 0.05 M phosphate buffer or 0.8% saline. Additionally, such pharmaceutically acceptable carriers can be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like.

“Administering” means delivering in a manner which is effected or performed using any of the various methods and delivery systems known to those skilled in the art. Administering can be performed, for example, topically, intravenously, pericardially, orally, via implant, transmucosally, transdermally, intramuscularly, subcutaneously, intraperitoneally, intrathecally, intralymphatically, intralesionally, or epidurally. Administering can also be performed, for example, once, a plurality of times, and/or over one or more extended periods.

A “subject” may be any animal, such as a mammal or a bird, including, without limitation, a cow, a horse, a sheep, a pig, a dog, a cat, a rodent such as a mouse or rat, a turkey, a chicken and a primate. In the preferred embodiment, the subject is a human being.

A “structural and functional homolog” of a chemical agent is one of a series of structurally and functionally similar agents, each of which is formed from the one before it by the addition of a constant element. A “structural and functional analog” of a chemical agent has a similar structure and function to that of the agent but differs from it in respect to a certain component or components. The term “analog” is broader than and encompasses the term “homolog.” “Analogs” also encompasses the following terms: “isomers” which are chemical compounds that have the same molecular formula but different molecular structures or different spatial arrangement of atoms; “prodrugs” which are functional derivatives of compounds that are readily convertible in vivo into the required compound; and “metabolites” which are the products of biological reactions and include active species produced upon introduction of chemical agents into an organism or other biological milieu.

Embodiments of the Invention

The present invention provides a method for treating a subject afflicted with atrial tachyarrhythmia comprising administering to the subject a therapeutically effective amount of an agent which inhibits protein kinase A (PKA) phosphorylation of a type 2 ryanodine receptor (RyR2) in the subject's heart, thereby treating the subject. In one embodiment, PKA phosphorylation of the RyR2 receptor causes dissociation of a FKBP12.6 binding protein from the RyR2 receptor. In another embodiment, the atrial tachyarrhythmia is an atrial fibrillation or a supra-ventricular tachyarrhythmia.

This invention also provides a method for treating a subject afflicted with atrial tachyarrhythmia comprising administering to the subject a therapeutically effective amount of an agent which inhibits dissociation of a FKBP12.6 binding protein from a type 2 ryanodine (RyR2) receptor in the subject's heart, thereby treating the subject. In one embodiment, the atrial tachyarrhythmia is an atrial fibrillation or a supra-ventricular tachyarrhythmia. In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999)

This invention further provides a method for treating a subject afflicted with atrial tachyarrhythmia comprising administering to the subject a therapeutically effective amount of an agent which mimics binding of a FKBP12.6 binding protein to a type 2 ryanodine receptor (RyR2) of the subject's heart, thereby treating the subject. In one embodiment, the atrial tachyarrhythmia is an atrial fibrillation or a supra-ventricular tachyarrhythmia.

Additionally, this invention is directed to an article of manufacture comprising (i) a packaging material having therein an agent which inhibits protein kinase A (PKA) phosphorylation of a type 2 ryanodine receptor (RyR2) and (ii) a label indicating a use for the agent in treating a subject afflicted with atrial tachyarrhythmia.

This invention is also directed to an article of manufacture comprising (i) a packaging material having therein an agent which inhibits dissociation of a FKBP12.6 binding protein from a type 2 ryanodine receptor (RyR2) and (ii) a label indicating a use for the agent in treating a subject afflicted with atrial tachyarrhythmia. In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

This invention is also directed to an article of manufacture comprising (i) a packaging material having therein an agent which mimics binding of a FKBP12.6 binding protein to a type 2 ryanodine receptor (RyR2) and (ii) a label indicating a use for the agent in treating a subject afflicted with atrial tachyarrhythmia.

The present invention additionally provides a method for inhibiting the onset of atrial tachyarrhythmia in a subject comprising administering to the subject a prophylactically effective amount of an agent which inhibits protein kinase A (PKA) phosphorylation of a type 2 ryanodine receptor (RyR2) in the subject's heart, thereby inhibiting the onset of an atrial tachyarrhythmia in the subject. In one embodiment, PKA phosphorylation of the RyR2 receptor causes dissociation of a FKBP12.6 binding protein from the RyR2 receptor. In another embodiment, the atrial tachyarrhythmia is an atrial fibrillation or a supra-ventricular tachyarrhythmia.

This invention also provides a method for inhibiting the onset of atrial tachyarrhythmia in a subject comprising administering to the subject a prophylactically effective amount of an agent which inhibits dissociation of a FKBP12.6 binding protein from a type 2 ryanodine (RyR2) receptor in the subject's heart, thereby inhibiting the onset of atrial tachyarrhythmia in the subject. In one embodiment, the atrial tachyarrhythmia is an atrial fibrillation or a supra-ventricular tachyarrhythmia. In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

This invention further provides method for inhibiting the onset of atrial tachyarrhythmia in a subject comprising administering to the subject a prophylactically effective amount of an agent which mimics binding of a FKBP12.6 binding protein to a type 2 ryanodine receptor (RyR2) of the subject's heart, thereby inhibiting the onset of atrial tachyarrhythmia in the subject. In one embodiment, the atrial tachyarrhythmia is an atrial fibrillation or a supra-ventricular tachyarrhythmia.

In addition, the present invention is directed to an article of manufacture comprising (i) a packaging material having therein an agent which inhibits protein kinase A (PKA) phosphorylation of a type 2 ryanodine receptor (RyR2) and (ii) a label indicating a use for the agent in inhibiting the onset of atrial tachyarrhythmia in a subject.

This invention is also directed to an article of manufacture comprising (i) a packaging material having therein an agent which inhibits dissociation of a FKBP12.6 binding protein from a type 2 ryanodine receptor (RyR2) and (ii) a label indicating a use for the agent in inhibiting the onset of atrial tachyarrhythmia in a subject. In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

This invention is further directed to an article of manufacture comprising (i) a packaging material having therein an agent which mimics binding of a FKBP12.6 binding protein to a type 2 ryanodine receptor (RyR2) and (ii) a label indicating a use for the agent in inhibiting the onset of atrial tachyarrhythmia in a subject.

This invention also provides a method for treating a subject afflicted with exercise-induced or stress-induced cardiac arrhythmia comprising administering to the subject a therapeutically effective amount of an agent which inhibits protein kinase A (PKA) phosphorylation of a type 2 ryanodine receptor (RyR2) in the subject's heart, thereby treating the subject. In one embodiment, PKA phosphorylation of the RyR2 receptor causes dissociation of a FKBP12.6 binding protein from the RyR2 receptor. In another embodiment, the cardiac arrhythmia is a ventricular fibrillation or a ventricular tachycardia. In a further embodiment, the subject is afflicted with catecholaminergic polymorphic ventricular tachycardia (CPVT).

This invention further provides a method for treating a subject afflicted with exercise-induced or stress-induced cardiac arrhythmia comprising administering to the subject a therapeutically effective amount of an agent which inhibits dissociation of a FKBP12.6 binding protein from a type 2 ryanodine (RyR2) receptor in the subject's heart, thereby treating the subject. In one embodiment, the cardiac arrhythmia is a ventricular fibrillation or a ventricular tachycardia. In another embodiment, the subject is afflicted with catecholaminergic polymorphic ventricular tachycardia (CPVT). In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

This invention also provides a method for treating a subject afflicted with exercise-induced or stress-induced cardiac arrhythmia comprising administering to the subject a therapeutically effective amount of an agent which mimics binding of a FKBP12.6 binding protein to a type 2 ryanodine receptor (RyR2) of the subject's heart, thereby treating the subject. In one embodiment, the cardiac arrhythmia is a ventricular fibrillation or a ventricular tachycardia. In another embodiment, the subject is afflicted with catecholaminergic polymorphic ventricular tachycardia (CPVT).

Additionally, this invention is directed to an article of manufacture comprising (i) a packaging material having therein an agent which inhibits protein kinase A (PKA) phosphorylation of a type 2 ryanodine receptor (RyR2) and (ii) a label indicating a use for the agent in treating a subject afflicted with exercise-induced or stress-induced cardiac arrhythmia.

This invention is also directed to an article of manufacture comprising (i) a packaging material having therein an agent which inhibits dissociation of a FKBP12.6 binding protein from a type 2 ryanodine receptor (RyR2) and (ii) a label indicating a use for the agent in treating a subject afflicted with exercise-induced or stress-induced cardiac arrhythmia. In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

This invention is further directed to an article of manufacture comprising (i) a packaging material having therein an agent which mimics binding of a FKBP12.6 binding protein to a type 2 ryanodine receptor (RyR2) and (ii) a label indicating a use for the agent in treating a subject afflicted with exercise-induced or stress-induced cardiac arrhythmia.

This invention also provides a method for inhibiting the onset of exercise-induced or stress-induced cardiac arrhythmia in a subject comprising administering to the subject a prophylactically effective amount of an agent which inhibits protein kinase A (PKA) phosphorylation of a type 2 ryanodine receptor (RyR2) in the subject's heart, thereby inhibiting the onset of exercise-induced or stress-induced cardiac arrhythmia in the subject. In one embodiment, PKA phosphorylation of the RyR2 receptor causes dissociation of a FKBP12.6 binding protein from the RyR2 receptor. In another embodiment, the cardiac arrhythmia is a ventricular fibrillation or a ventricular tachycardia. In a further embodiment, the subject is afflicted with catecholaminergic polymorphic ventricular tachycardia (CPVT).

This invention further provides a method for inhibiting the onset of exercise-induced or stress-induced cardiac arrhythmia comprising administering to the subject a prophylactically effective amount of an agent which inhibits dissociation of a FKBP12.6 binding protein from a type 2 ryanodine (RyR2) receptor in the subject's heart, thereby inhibiting the onset of exercise-induced or stress-induced cardiac arrhythmia in the subject. In one embodiment, the cardiac arrhythmia is a ventricular fibrillation or a ventricular tachycardia. In another embodiment, the subject is afflicted with catecholaminergic polymorphic ventricular tachycardia (CPVT). In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

This invention also provides a method for inhibiting the onset of exercise-induced or stress-induced cardiac arrhythmia comprising administering to the subject a prophylactically effective amount of an agent which mimics binding of a FKBP12.6 binding protein to a type 2 ryanodine receptor (RyR2) of the subject's heart, thereby inhibiting the onset of exercise-induced or stress-induced cardiac arrhythmia in the subject. In one embodiment, the cardiac arrhythmia is a ventricular fibrillation or a ventricular tachycardia. In another embodiment, the subject is afflicted with catecholaminergic polymorphic ventricular tachycardia (CPVT).

In addition, the present invention is directed to an article of manufacture comprising (i) a packaging material having therein an agent which inhibits protein kinase A (PKA) phosphorylation of a type 2 ryanodine receptor (RyR2) and (ii) a label indicating a use for the agent in inhibiting the onset of exercise-induced or stress-induced cardiac arrhythmia in a subject.

This invention is also directed to an article of manufacture comprising (i) a packaging material having therein an agent which inhibits dissociation of a FKBP12.6 binding protein from a type 2 ryanodine receptor (RyR2) and (ii) a label indicating a use for the agent in inhibiting the onset of exercise-induced or stress-induced cardiac arrhythmia in a subject. In different embodiments, the agent is JTV-519 (also known as K201) or any other compound in this class of compounds that are derivatives of 1,4-benzothiazepine (Yano et al., 2003; Kaneko, 1994; Hachida et al., 1999; Kimura et al., 1999).

Finally, this invention is directed to an article of manufacture comprising (i) a packaging material having therein an agent which mimics binding of a FKBP12.6 binding protein to a type 2 ryanodine receptor (RyR2) and (ii) a label indicating a use for the agent in inhibiting the onset of exercise-induced or stress-induced cardiac arrhythmia in a subject.

Approaches to designing and synthesizing receptor-selective compounds are well known and include traditional medicinal chemistry and the newer technology of combinatorial chemistry, both of which are supported by computer-assisted molecular modeling. With such approaches, chemists and pharmacologists use their knowledge of the structures of the targeted receptor subtype and compounds determined to bind and/or activate or inhibit activation of the receptor to design and synthesize structures that will have activity at these receptor subtypes.

Combinatorial chemistry involves automated synthesis of a variety of novel compounds by assembling them using different combinations of chemical building blocks. The use of combinatorial chemistry greatly accelerates the process of generating compounds. The resulting arrays of compounds are called libraries and are used to screen for compounds (“lead compounds”) that demonstrate a sufficient level of activity at receptors of interest. Using combinatorial chemistry it is possible to synthesize “focused” libraries of compounds anticipated to be highly biased toward the receptor target of interest.

Once lead compounds are identified, whether through the use of combinatorial chemistry or traditional medicinal chemistry or otherwise, a variety of homologs and analogs are prepared to facilitate an understanding of the relationship between chemical structure and biological or functional activity. These studies define structure activity relationships which are then used to design drugs with improved potency, selectivity and pharmacokinetic properties. Combinatorial chemistry is also used to rapidly generate a variety of structures for lead optimization. Traditional medicinal chemistry, which involves the synthesis of compounds one at a time, is also used for further refinement and to generate compounds not accessible by automated techniques. Once such drugs are defined the production is scaled up using standard chemical manufacturing methodologies utilized throughout the pharmaceutical and chemistry industry.

This invention will be better understood from the Experimental Details which follow. However, one skilled in the art will readily appreciate that the specific methods and results discussed are merely illustrative of the invention as described more fully in the claims which follow thereafter.

Experimental Details

Experimental Set I

Materials and Methods

Immunoprecipitation and Back-Phosphorylation of RyR2

Homogenates and sarcoplasmic reticulum (SR) membranes were prepared from cardiac ventricular tissue as described by Kaftan et al. (1996). Cardiac SR (200 μg) or homogenates (500 μg) were suspended in 0.5 ml of buffer (50 mM Tris-HCl (pH 7.4), 0.9% NaCl, 0.5 mM NaF, 0.5 mM Na₃VO₄, 0.25% Triton X100, and protease inhibitors). Samples were incubated overnight at 4° C. with the antibodies indicated below (immunoglobulin G (IgG) alone was used as a negative control for immunoprecipitations with each antibody, data not shown). Protein A-Sepharose beads were added to the samples followed by incubation at 4° C. for 1 hour with constant mixing. Beads were washed with a 1× phosphorylation buffer (8 mM MgCl, 10 mM ethylene glycol-bis(β-aminoethylether)N,N,N′,N′-tetraacetic acid (EGTA), and 50 mM Tris/piperazine-N,N′-bis(2-ethanesulfonic acid), pH 6.8), resuspended in 10 μl of a 1.5× phosphorylation buffer containing either vehicle alone, catalytic subunit of PKA (Sigma, St. Louis, Mo.), or PKA plus a PKA inhibitor (PKI₅₋₂₄, 500 nM, Calbiochem, San Diego, Calif.), or cAMP as indicated. Back-phosphorylation of immunoprecipitated RyR2 was initiated by addition of PKA (5 units) and MgATP (33 μM) and terminated after incubation for 5 minutes at room temperature by the addition of 5 μl of stop solution (4% sodium dodecylsulfate (SDS) and 0.25 M dithiothreitol (DTT)). RyR2 phosphorylation with: 1) protein kinase C (PKC) (0.05 units, Calbiochem) was performed under similar conditions (1.5 mM CaCl₂ was added to the phosphorylation buffer); and 2) with Ca²⁺/calmodulin-dependent protein kinase (CaMKII, 0.2 μg, Upstate Biotech, Lake Placid, N.Y.); 1.5 mM CaCl₂ and 5 μM calmodulin were added to the phosphorylation buffer. In some experiments the adenosine triphosphate (ATP) solution also contained 10% [γ³²P]-ATP (NEN Life Sciences, Boston, Mass.). Samples were heated to 95° C. and size fractionated on 6% SDS-PAGE. The radioactive signal corresponding to the RyR2 band was quantified using a Molecular Dynamics Phosphorimager and ImageQuant software (Amersham Pharmacia Biotech, Piscataway, N.J.). Back-phosphorylation was quantified using a phosphorimager, non-specific phosphorylation (not inhibited by addition of the PKA inhibitor) was subtracted and the resulting value was divided by the amount of RyR2 protein in each immunoprecipitate (determined by immunoblotting and densitometry or by [³H] ryanodine binding) and expressed as the inverse of the PKA-dependent [γ³²P]-ATP signal. Microcystin-Sepharose (35 μl, UBI) was used to isolate RyR2 from 200 μg of cardiac SR by incubating at 4° C. for 1 hour followed by washing. Beads were resuspended in 6X SDS loading buffer, boiled and the supernatant was size fractionated on SDS-PAGE. Phosphatases PP1 and PP2A bound to the microcystin-Sepharose beads were competed off with addition of free microcystin-LR (Calbiochem).

Stoichiometry of PKA Phosphorylation

Maximum PKA-dependent phosphorylation was determined by pre-treatment of RyR2 with alkaline phosphatase (AP, 1:100 enzyme:protein, New England Biolabs) for 20 minutes at 37° C. to remove bound phosphate. The reaction was terminated by the addition of 5 μl of stop solution. NaF was omitted, then added after dephosphorylation to terminate the reaction. Samples were back-phosphorylated with PKA as described above. To calculate the stoichiometry of PKA phosphorylation of RyR2 a phosphorimager was used to calibrate signals generated by [γ³²P]-ATP standards of known specific activity (from 2.0×10⁻⁴ to 2.0×10⁻³ μCi/μl). The molar ratio of ³²P/RyR2 was calculated by dividing the ³²P-phosphorylation by the amount of high affinity [³H]ryanodine binding (one high affinity ryanodine binding site per RyR2) in each sample of immunoprecipitated RyR2 protein.

Immunoblots

Immunoblots were performed as described (Moschella and Marks, 1993) using the following antibodies: anti-FKBP12 (1:1000), anti-RyR (5029, 1:3000) (Jayaraman et al., 1992), anti-PP1 (1:1000), anti-PP2A (1:1000), anti-CnA (1:1000), anti-PKA catalytic subunit (1:1000, Transduction Labs, Lexington, Ky.), anti-phosphoserine (1 μg/ml, Zymed San Francisco, Calif.), anti-mAKAP (3 μg/ml, Upstate Biotechnology, Lake Placid, N.Y.), or purified VO56 (anti-mAKAP antibody) (Kapiloff et al., 1999). After washing, membranes were incubated with peroxidase conjugated goat anti-rabbit or goat anti-mouse IgG antiserum (1:3000, Boehringer-Mannheim) for 60 minutes at room temperature, washed X3 with Tris-buffered saline (TBS), 0.1% Tween 20, and developed using enhanced chemiluminescence (ECL, Amersham).

Yeast Two-Hybrid Assay to Identify the FKBP12.6 Binding Site

Human FKBP12.6 cDNA was subcloned into the yeast two hybrid vector pEG202 (OriGene Technologies, Rockville, Md.) to make pEGFKBP12.6 (FKBP12.6 fused to the GAL4 DNA binding domain). Human RyR2 cDNA fragments subcloned into the yeast two hybrid vector pJG4-5 (OriGene) were confirmed by sequencing. The yeast two hybrid assay for protein-protein interaction was performed using the DupLEX-A yeast system (OriGene) per manufacturer's instructions. pEGFKBP12.6 and pAD-GAL4RyR2/2361-2496 were co-transformed into the rapamycin-resistant mutant yeast strain Y663 (Lorenz and Heitman, 1995), and liquid β-galactosidase assays were performed in the absence or presence of rapamycin (0.1, 1.0 and 10 μM) which competes with RyR2 for binding to FKBP12.6.

Site-Directed Mutagenesis, Expression of GST-RyR2 Fusion Proteins and In Vitro Mapping

pGST-RyR2 constructs were generated using rabbit or human RyR2 cDNA, and fusion proteins were expressed and purified with glutathione Sepharose beads. Site-directed mutagenesis was performed using the 5′ Prime-3′-Prime Site-directed Mutagenesis Kit (Amersham Pharmacia Biotech) as per manufacturer's instructions. pGST-RyR2 fusion proteins bound to Sepharose beads were incubated with canine cardiac SR (200 μg protein), pelleted, washed with modified RIPA buffer, size fractionated on SDS-PAGE, and immunoblotted with the indicated antibodies.

Immunohistochemistry

Human cardiac tissue was fixed in 10% neutral buffered formalin, and embedded in paraffin. Sections (4 μM) were dried overnight at 37° C., de-waxed with xylenes, re-hydrated, incubated with phosphate buffered saline (PBS), 0.2% Tween-20 for 5 minutes, then incubated with 5% goat serum in PBS for 1 hour at room temperature. Sections were then incubated with either pre-immune rabbit serum (IgG) or primary antibody [MAKAP (VO56), RyR2 (monoclonal, Affinity Bioreagents); 1:50] in PBS, 3% bovine serum albumin (BSA) overnight at 4° C., followed by intensive washing with PBS. Sections were then incubated with either FITC or rhodamine secondary antibody (1:100; Zymed) in 3% BSA-PSA for 1 hour at room temperature, washed with PBS and stained with Hoescht dye (10 μg/ml) for 5 minutes, followed by intensive PBS washing. For double immunostaining, slides were sequentially stained with two individual primary antibodies followed by simultaneous incubation with the secondary antibodies. Immunostained slides were examined using a Nikon microscope with 100× objective; images were acquired with a SPOT RT camera (Diagnostic Instruments Inc) using Adobe Photoshop.

Isolation of RyR2 and Single Channel Recordings

Cardiac muscle heavy SR was incubated with [³H] ryanodine, solubilized with 3-[(3-cholamidopropyl)-dimethylammonio]-1-propanesulfonate (CHAPS) and centrifuged for 14 hours on a 10 to 32% linear sucrose gradient at 26,000 rpm, 2° C. in a Sorvall AH-629 rotor (Brillantes et al., 1994; Marx et al., 1998). Single channel recordings were performed as described (Brillantes et al., 1994). Single channel properties were evaluated using pCLAMP 6.02 software (Axon Instruments). Open probabilities were determined by analyzing data at 10 and 30 second intervals over a minimum of 3 minutes. At the conclusion of each experiment ryanodine and/or ruthenium red were applied to confirm the identity of channels as ryanodine receptors. Results are presented as mean±standard deviation. The Student's t-test was used for statistical analyses of the dwell time distributions and open probabilities.

Human Heart Samples and Left Ventricular Assist Device (LVAD).

The use of human tissues for this study was approved by the Institutional Review Board of Columbia-Presbyterian Medical Center. Normal and failing human heart tissues were obtained as previously described from patients undergoing cardiac transplant (Go et al., 1995). Left ventricular assist devices (Thermo Cardiosystems Inc., Woburn, Mass.) were implanted in patients as a bridge to heart transplantation according to standard clinical practice (Frazier, 1994).

Muscle Strip Function

Trabeculae (diameters <1 mm, lengths >3 mm) were obtained from human left ventricular apical core samples obtained at the time of LVAD implantation or from hearts explanted at the time of orthotopic cardiac transplantation. Trabeculae were placed in a standard muscle bath, attached to a force transducer and stimulated at 1 Hz, left to equilibrate for 1 hour prior to study at slack length, then stretched progressively to the point of maximal tension development (Lₘₐₓ). β-adrenergic response was tested by superfusing the muscle with Krebs-Ringer solution containing isoproterenol (4 μM).

Canine Heart Failure Model

Canine heart failure was induced by rapid cardiac pacing at 210 beats/minute for 3 weeks followed by an additional week of pacing at 240 beats/minute as described previously (Wang et al., 1997). This rapid cardiac pacing regimen induces severe heart failure as evidenced by an average 40% reduction in left ventricular dP/dtₘₐₓ (to ˜1800 mmHg/second), 20% reductions in peak left ventricular and mean aortic pressures (to 100 and 85 mm Hg, respectively), a 50% increase in resting heart rate (to 132 beats/minute), and a rise in end-diastolic pressure to greater than 20 mm Hg (Wang et al., 1997). All procedures were approved by the Institutional Animal Care Committee.

Methods of Transfecting Cells

Methods of transfecting cells with nucleic acid encoding a ryanodine receptor to obtain cells in which the ryanodine receptor is expressed are well known in the art (see, for example, Brillantes et al., 1994). In non-muscle cells, the RyR2 receptor is expressed on the endoplasmic reticulum (ER). The cells may be additionally transfected with nucleic acid encoding a β-AR to obtain cells in which both the RyR2 receptor and β-AR are expressed. Such transfected cells may also be used to test chemical agents and screen libraries of chemical agents to obtain agents that bind receptors as well as agents that activate or inhibit activation of functional responses in such cells, and therefore are likely to do so in vivo.

A broad variety of host cells can be used to study heterologously expressed proteins. In addition to HEK293 cells, these cells include, but are not limited to, mammalian cell lines such as Chinese hamster ovary (CHO), COS-7, mouse embryonic fibroblast NIH-373, LM(tk⁻), mouse Y1 and HeLa cells; insect cell lines such as Sf9, Sf21 and Trichoplusia ni 5B-4 cells; amphibian cells such as Xenopus oocytes and Xenopus melanophore cells; assorted yeast strains; assorted bacterial cell strains; and others. Culture conditions for each of these cell types is specific and is known to those familiar with the art.

DNA encoding proteins to be studied can be transiently expressed in a variety of mammalian, insect, amphibian, yeast, bacterial and other cells lines by several transfection methods including, but not limited to, calcium phosphate-mediated, DEAE-dextran-mediated, liposomal-mediated, viral-mediated, electroporation-mediated, and microinjection delivery. Each of these methods may require optimization of experimental parameters depending on the DNA, cell line, and the type of assay to be subsequently employed.

Heterologous DNA can be stably incorporated into host cells, causing the cell to perpetually express a foreign protein. Methods for the delivery of the DNA into the cell are similar to those described above for transient expression but require the co-transfection of an ancillary selectable marker gene to confer a selectable phenotype, e.g., drug resistance, to the targeted host cell. The ensuing drug resistance can be exploited to select and maintain cells that have taken up the DNA. A variety of resistance genes are available including but not restricted to kanamycin, G-418 and hygromycin.

RyR2 Binding Assays

Methods of conducting binding assays are well known in the art. Labeled chemical agents are placed in contact with intact cells, or a cell extract containing SR or ER, expressing the RyR2 receptor. Methods of preparing a cell extract containing SR or ER are known in the art (e.g., Kaftan et al., 1996). If the agent is labeled with a radioactive isotope such as ³H, ¹⁴C, ¹²⁵I, ³⁵S, ³²P or ³³P, the bound agent may be detected using liquid scintillation counting, scintillation proximity or any other method of detection for radioactive isotopes. If the agent is labeled with a fluorophore, the bound labeled agent may be measured by methods such as, but not restricted to, fluorescence intensity, time-resolved fluorescence, fluorescence polarization, fluorescence transfer, or fluorescence correlation spectroscopy. If the agent is labeled with a dye, the bound labeled agent may be measured by various methods including colorimetry or spectrophotometry. If the agent is tagged with a chemiluminescent label, the bound labeled agent may be measured, for example, using a luminometer with a photomultiplier tube or avalanche photodiode. In addition to detecting a labeled agent bound to the RyR2 receptor, the binding of a nonlabeled, second agent to the receptor may be assayed by its competitive inhibition of the binding of a labeled, first agent that is known to bind specifically to the receptor.

Assays for Compounds to Treat Heart Disease

PKA phosphorylation of RyR2 increases the activity of the RyR2 channel resulting in the release of more calcium into the cytoplasm of the cell for a given activator of the channel. Compounds that block PKA activation of RyR2 would be expected to reduce the activation of the RyR2 channel resulting in less release of calcium into the cell. Compounds that bind to the RyR2 channel at the FKBP12.6 binding site but do not come off the channel when the channel is phosphorylated by PKA would also be expected to decrease the activity of the channel in response to PKA activation or other triggers that activate the RyR2 channel. Such compounds would also result in less calcium release into the cell.

One assay for compounds that may be effective in treating heart disease involves measuring the release of calcium into cells via the RyR2 channel using calcium-sensitive fluorescent dyes (e.g., Fluo-3, Fura-2). The assay involves loading cells with the fluorescent dye and stimulating the cells with a RyR2 activator and determining whether or not a compound added to the cells reduces the calcium-dependent fluorescent signal (Brillantes et al., 1994; Gillo et al, 1993; Jayaraman et al., 1996). One RyR2 activator is caffeine which can be added to the cell. When calcium is released into the cytoplasm of the cell it is bound by the calcium-sensitive dye which then emits a fluorescent signal. Calcium-dependent fluorescent signals are monitored with a photomultiplier tube and analyzed with appropriate software as described by Brillantes et al., 1994; Gillo et al., 1993; and Jayaraman et al., 1996. This assay can be easily automated to screen large numbers of compounds using multiwell dishes. The assay involves expressing recombinant RyR2 channels in a heterologous expression system such as bacterial, yeast, insect, Sf9, HEK293, CHO, COS-7, LM(tk-), mouse embryonic fibroblast NIH-3T3, 293 human embryonic kidney, or HeLa cells (Brillantes et al., 1994). In non-muscle cells, the RyR2 receptor is expressed on the endoplasmic reticulum. When the RyR2 channel is activated, calcium is released from the endoplasmic reticulum into the cytoplasm of the cell. RyR2 receptors could be co-expressed with beta adrenergic receptors. This would permit the assessment of compounds on RyR2 receptor activation in response to addition of beta adrenergic receptor agonists.

Another assay involves measuring the level of protein kinase A phosphorylation of RyR2 which correlates with the degree of heart failure and can be used to determine the efficacy of compounds designed to block the protein kinase A phosphorylation of the RyR2 channel. This assay can be used in connection with animal models in which heart failure is induced by rapid cardiac pacing. The assay is based on the use of antibodies that are specific for the RyR2 channel protein (anti-RyR2 antibody). For this assay the RyR2 channel protein is immunoprecipitated with the anti-RyR2 antibody and then back-phosphorylated with protein kinase A and [γ³²P]-adenosine triphosphate (ATP). The amount of radioactive ³²P label that is transferred to the RyR2 receptor protein can be measured using a phosphorimager. In another version of the assay, the antibody is specific for the phosphorylated form of the RyR2 receptor, in which case back-phosphorylation is not necessary.

Other assays for RyR2 receptor channel function involve measuring the degree of association of the FKBP12.6 binding protein with the RyR2 receptor, the subconductance state of the RyR2 receptor channel, the Ca²⁺ sensitivity for activation of the RyR2 receptor channel, or the open probability (Pₒ) of the RyR2 receptor channel.

Results

Protein Kinase A Phosphorylates RyR2

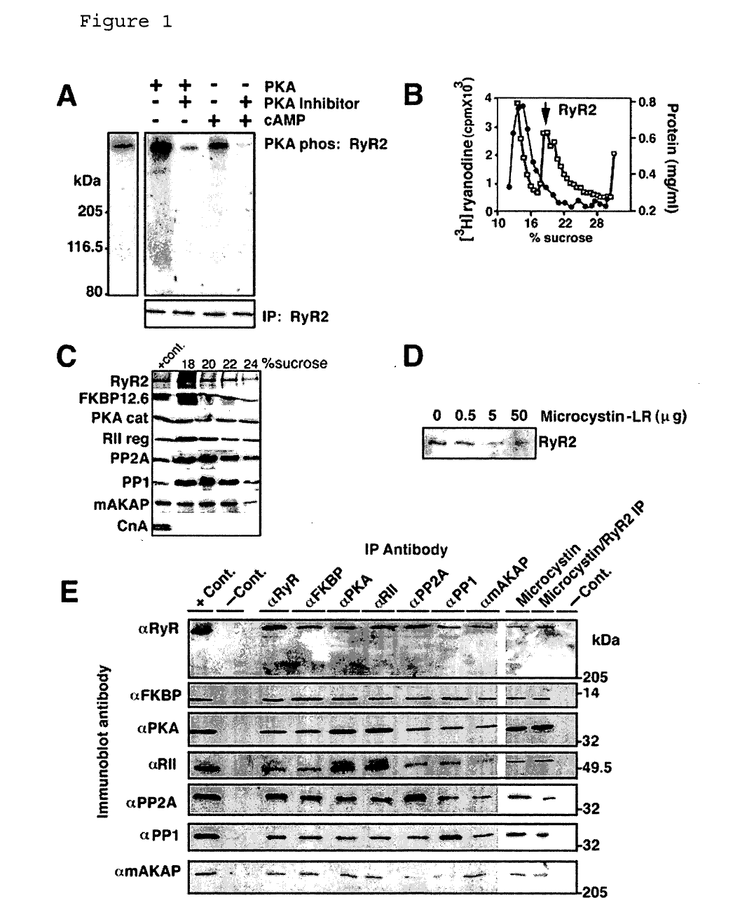

The 565,000 dalton RyR2 polypeptide was PKA phosphorylated in in vitro kinasing reactions (FIG. 1A). To confirm the identity of the PKA phosphorylated high molecular weight protein as RyR2, the phosphorylated band was immunoblotted with anti-RyR antibody. The specificity of the phosphorylation was demonstrated using a PKA inhibitor (FIG. 1A). Addition of phosphorylation buffer including cAMP without exogenous PKA also resulted in phosphorylation of RyR2 that was inhibited by PKI indicating that endogenous PKA was associated with RyR2 (FIG. 1A). The stoichiometry of PKA phosphorylation was determined by immunoprecipitating RyR2 from cardiac muscle SR, fully dephosphorylating the RyR2 protein with alkaline phosphatase, and then phosphorylating with PKA and [γ³²P]-ATP. The stoichiometry of PKA phosphorylation was 3.8±0.1 moles of phosphate per mole of channel (each channel comprises four RyR2 subunits) or about one mole of phosphate per RyR2 subunit indicating that each RyR2 protein is PKA phosphorylated on a single amino acid residue.

RyR2 Macromolecular Complex Includes FKBP12.6, PKA, PP1, PP2A and mAKAP

RyR2 was isolated by sucrose density gradient centrifugation using [³H] ryanodine (FIG. 1B) as described (Marx et al., 1998). Individual tetrameric RyR2 channels sediment as 30S complexes and multiple channels (two or more) sediment as denser complexes (Marx et al., 1998). The muscle A kinase anchoring protein (mAKAP) that binds PKA and targets it to substrates has been localized to cardiac SR (Kapiloff et al., 1999; Yang et al., 1998). The major protein phosphatases in cardiac muscle are protein phosphatase 2A (PP2A), protein phosphatase 1 (PP1) (MacDougall et al., 1991), and calcineurin (CnA). Fractions from the sucrose gradient were immunoblotted with either anti-RyR antibody or with antibodies that recognize FKBP12.6, the catalytic subunit of PKA, the PKA regulatory subunit (RII), PP2A, PP1, mAKAP or CnA (FIG. 1C) all of which (with the exception of CnA) were detected in all fractions containing RyR2. These data are consistent with a high molecular weight complex comprised of RyR2, FKBP12.6, PKA, RII, PP1, PP2A and mAKAP.

The phosphatase inhibitor microcystin binds to PP1 and PP2A. RyR2 was sedimented by binding to microcystin-Sepharose beads, and the specificity of this interaction was demonstrated by competing off RyR2 using free microcystin-LR (FIG. 1D). Co-immunoprecipitations were performed showing that FKBP12.6, PKA, RII, PP2A, PP1 and mAKAP all co-immunoprecipitated with RyR2, indicating physical association of these proteins and the SR Ca²⁺ release channel (FIG. 1E). The existence of a macromolecular complex was shown independently by first sedimenting the complex with microcystin-Sepharose beads followed by competing the complex off from the beads with free microcystin-LR and then immunoprecipitating each of the components of the complex (FIG. 1E, last three lanes). Taken together these data show that FKBP12.6, PKA, RII, PP1, PP2A and mAKAP comprise a macromolecular complex with RyR2.

PKA Hyperphosphorylation of RyR2 in Failing Heart Muscle

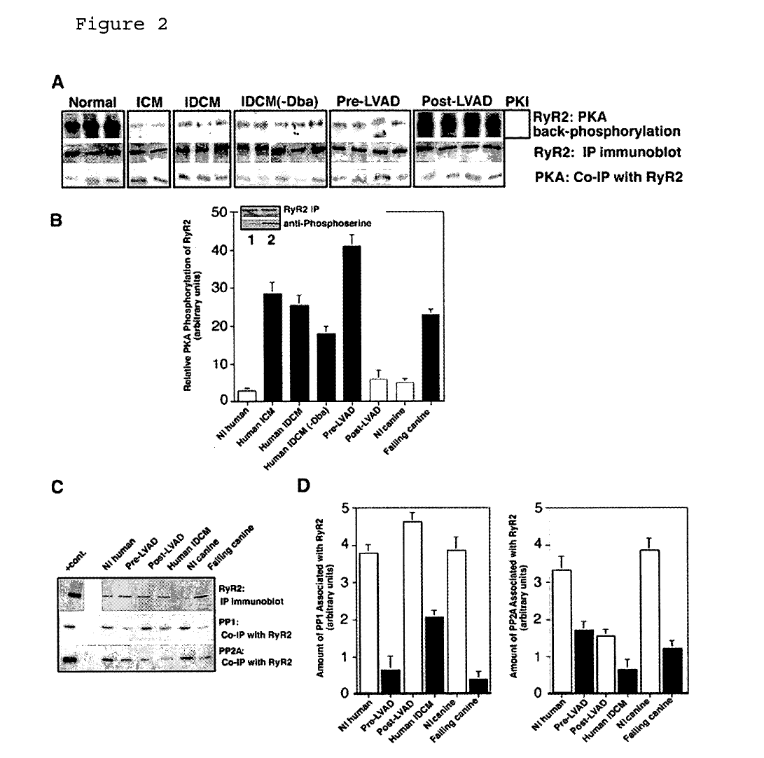

Increased sympathetic activity is an important physiologic response to stress resulting in activation of the adrenergic signaling pathway that generates increased cAMP levels and activates PKA. In failing hearts (regardless of the etiology of the damage to the heart) circulating catecholamine levels are markedly increased. Specific PKA phosphorylation of RyR2 in normal and failing hearts was examined using both back phosphorylation with [γ³²P]-ATP and anti-phosphoserine immunoblots (FIGS. 2A and B).

Strikingly, PKA phosphorylation of RyR2 was significantly elevated in failing hearts from humans and from animal models (dogs with pacing-induced heart failure) compared to non-failing hearts (FIGS. 2A and 2B). PKA phosphorylation of RyR2 channels from failing hearts was increased ˜4-fold compared to RyR2 channels from non-failing hearts. The stoichiometry of PKA back-phosphorylation of RyR2 channels isolated from failing hearts was 0.7±0.3 moles of phosphate transferred per mole of channel (n=8) compared to 3.1±0.1 moles of phosphate transferred per mole of channels from normal non-failing hearts, (n=6, p<0.0001) These data suggest that in failing hearts approximately three of the four PKA sites on the tetrameric RyR2 channel are phosphorylated in vivo, whereas only one site is phosphorylated in vivo on RyR2 isolated from normal non-failing hearts.

This increase in PKA phosphorylation of RyR2 was not due to an increase in the levels of PKA protein associated with RyR2 in failing hearts as determined by co-immunoprecipitation of PKA with RyR2 (FIG. 2A). PKA back-phosphorylation was performed using immunoprecipitated RyR2 to ensure that the phosphorylation signal which was measured represented specifically RyR2 PKA phosphorylation. RyR2 levels are decreased in failing hearts (Go et al., 1995). PKA phosphorylation of RyR2 was normalized to the amount of immunoprecipitated RyR2 protein to enable valid comparisons of the amount of PKA phosphorylation per RyR2 molecule from normal and failing hearts (FIGS. 2A and 2B). Moreover, identical results were obtained when immunoprecipitated RyR2 was immunoblotted with an anti-phosphoserine antibody (e.g., see FIG. 2B inset) confirming that the RyR2 channels from failing hearts were PKA hyperphosphorylated compared to channels from non-failing hearts.

Left ventricular assist devices (LVADs) are used as a bridge to transplantation when donor hearts are not available. Studies have shown that the hemodynamic unloading of the left ventricle provided by LVADs results in a significant improvement in cardiac contractile function when the device is implanted in failing hearts (Levin et al., 1995). At the time of LVAD insertion a tissue core is removed from the patient's left ventricle and this tissue can then be compared to tissue from the explanted heart which becomes available at the time of transplantation. Thus, the pre-LVAD sample comes from failing hearts and the post-LVAD sample comes from hearts with improved function. PKA phosphorylation of RyR2 was significantly increased in pre-LVAD heart samples compared to samples from non-failing hearts and returned to normal levels following LVAD treatment (FIGS. 2A and 2B). Taken together these data show: 1) PKA phosphorylation of RyR2 is regulated physiologically in vivo; 2) heart failure is associated with increased PKA phosphorylation of RyR2; and 3) the PKA phosphorylation of RyR2 returns to normal levels when the cardiac function is improved by LVAD insertion.

Many patients with end-stage heart failure are treated with β-adrenergic agonists (e.g., dobutamine) prior to cardiac transplantation, while some patients are admitted directly from home when a donor heart becomes available and therefore are not receiving β-adrenergic agonists which are administered intravenously in the hospital. PKA phosphorylation of RyR2 was significantly elevated in the hearts from patients not on β-adrenergic agonists compared to normals (FIGS. 2A and B). PKA phosphorylation of RyR2 was significantly further increased in hearts from those patients treated with β-adrenergic agonists prior to cardiac transplantation (FIGS. 2A and 2B). These data indicate that exogenous administration of β-adrenergic agonists to patients with heart failure can further increase the PKA phosphorylation of RyR2 in the heart.

To determine whether the increased PKA phosphorylation of RyR2 observed in failing hearts was explained solely by an increase in PKA activity or possibly by a concomitant decrease in the activity of phosphatases which catalyze the removal of phosphate groups, the amounts of PP1 and PP2A physically associated with RyR2 in failing hearts were compared to those in normal hearts from humans and dogs (FIGS. 2C and D). There was a significant decrease in the levels of PP1 and PP2A that co-immunoprecipitated with RyR2 from failing hearts (FIGS. 2C and D). The decrease in the amount of PP1 (but not of PP2A) associated with RyR2 was restored to normal by LVAD treatment (FIGS. 2C and 2D). These data suggest that at least in part the PKA hyperphosphorylation of RyR2 is due to a decrease in phosphatase bound to the RyR2 channel macromolecular complex.

Mapping Signaling Complex Binding Sites on RyR2

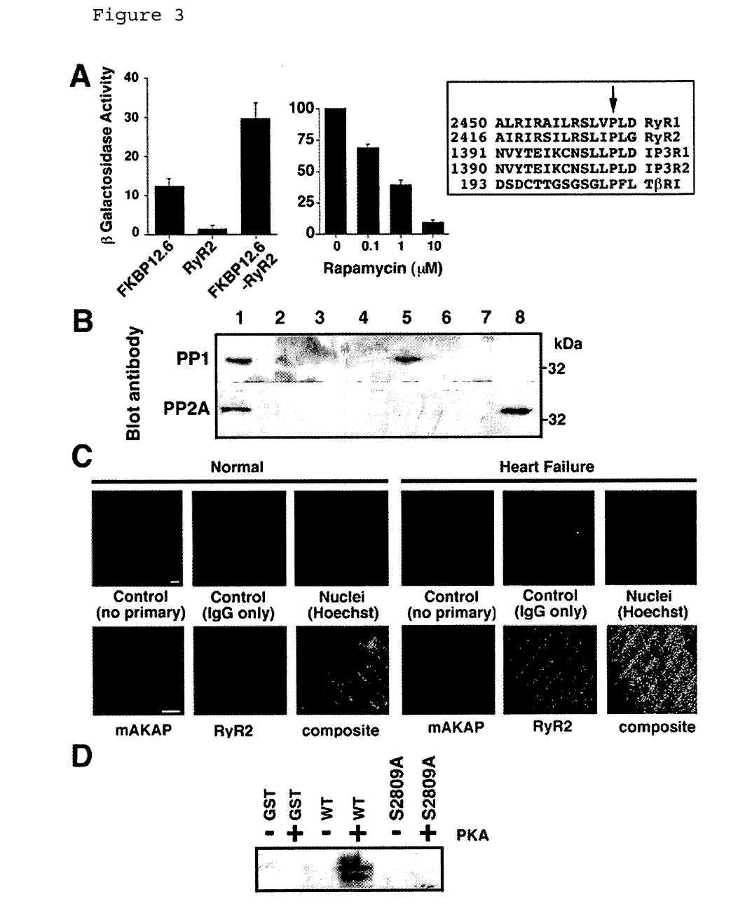

The FKBP12.6 binding site on RyR2 was identified using a yeast two-hybrid protein interaction screen in which yeast were transformed with vectors containing either fragments of RyR2 or the full length FKBP12.6 fused to the Gal4 activation domain or DNA binding domain. One RyR2 fragment corresponding to amino acid residues 2361-2496 (Otsu et al., 1990) resulted in a positive interaction with FKBP12.6 as determined by an increase in β-galactosidase activity (FIG. 3A). Using rapamycin-resistant yeast (Lorenz and Heitman, 1995), rapamycin was shown to specifically inhibit the interaction between FKBP12.6 and RyR2 (FIG. 3A) in yeast in a concentration-dependent manner indicating that the interaction between FKBP12.6 and RyR2 was specific. This fragment contains the hydrophobic motif comprised of isoleucine 2427 and proline 2428 (FIG. 3A) that is homologous to the FKBP12 binding site in RyR1, IP3R1 (Cameron et al., 1997), and TRI (FIG. 3A). Using GST-RyR2 fusion proteins in pull-down assays with cardiac SR, binding domains on RyR2 for PP1 (residues 513-808), and PP2A (residues 1451-1768) were mapped (FIG. 3B). Interestingly, the binding domains for PP1 and PP2A both contain leucine/isoleucine zippers. Immunohistochemistry showed that mAKAP is present in the cardiac SR, the same cellular location as RyR2, and there was no difference in this regard between normal and failing human hearts (FIG. 3C). Wild type and mutant GST-RyR2 fusion proteins were used to determine the site of PKA phosphorylation (serine 2809, FIG. 3D).

PKA Phosphorylation of RyR2 Inhibits FKBP12.6 Binding

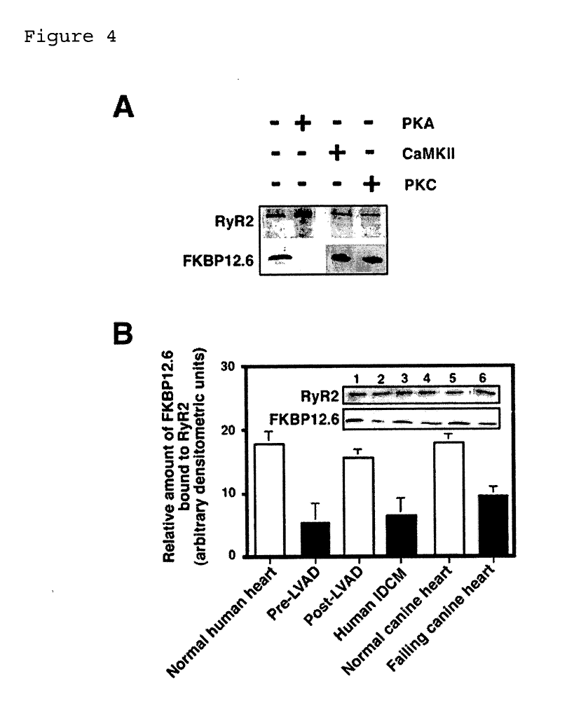

PKA phosphorylation of immunoprecipitated RyR2 (there was no PKA phosphorylation of FKBP12.6) resulted in a significant decrease (˜90±9% reduction, n=8, p<0.001) in the amount of FKBP12.6 co-immunoprecipitating with RyR2 (FIG. 4A). No dissociation of FKBP12.6 from RyR2 was observed in the following negative controls: 1) including the PKA inhibitor PKI in the reaction; 2) boiling the PKA; 3) omitting ATP. Neither Ca²⁺ calmodulin kinase (CaMKII) nor protein kinase C (PKC), both of which phosphorylate RyR2, caused the dissociation of FKBP12.6 from RyR2 indicating that the PKA phosphorylation-induced dissociation of FKBP12.6 from RyR2 is a specific effect (FIG. 4A). Furthermore, there was a significant decrease in the amount of FKBP12.6 that co-immunoprecipitated with RyR2 from failing hearts compared to normal hearts both in humans (65±11% reduction, n=4, p<0.005) and in dogs with pacing-induced heart failure (50±8% reduction, n=3, p<0.005) (FIG. 4B). The total amount of FKBP12.6 was the same in homogenates from normal and failing hearts as determined by immunoblotting (data not shown). These data show that PKA phosphorylation of RyR2 provides a mechanism for the physiologic and pathophysiologic regulation of FKBP12.6 binding to RyR2.

PKA Phosphorylation of RyR2 Increases Pₒ and Induces Subconductance States

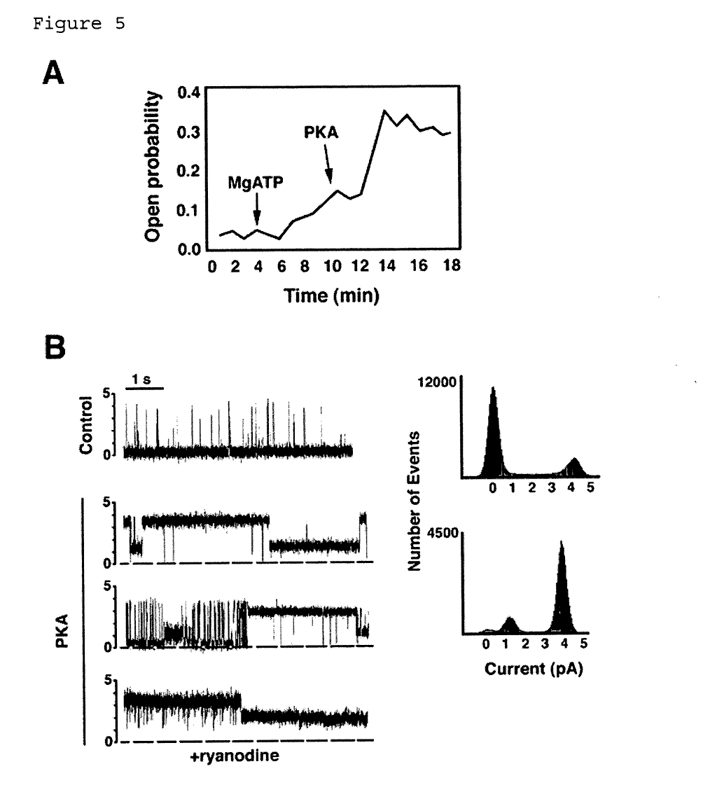

The dissociation of FKBP12/12.6 from RyR1 or RyR2 has previously been shown to increase the channel open probability (Pₒ) by shifting the Ca²⁺-dependence for activation to the left (Brillantes et al., 1994; Kaftan et al., 1996). A second effect of dissociation of FKBP12/12.6 from the channels is to induce subconductance states consistent with a destabilizat‡ion of the tetrameric channel structure (Brillantes et al., 1994). PKA phosphorylation of RyR2 in planar lipid bilayers resulted in a significant increase in Po from 0.10±0.03 to 0.35±0.06 (n=4/4, p<0.001, e.g. FIG. 5A). PKA phosphorylation of RyR2 also induced subconductance states (n=4, e.g. FIG. 5B) similar to those seen after dissociation of FKBP from the RyR channels (Brillantes et al., 1994; Kaftan et al., 1996; Marx et al., 1998). PKA phosphorylation of RyR2 channels did not alter the mean open time of the full conductance state (for control channels τₒ=2.1±0.8 ms vs. 2.6±0.6 ms following PKA treatment, n=4, p=NS). However, long lasting subconductance states (τₒ=502.1±40.8 ms) were observed following PKA phosphorylation of RyR2 channels in the bilayer (FIG. 5B). In addition, the phosphatase inhibitor okadaic acid (1 μM) significantly increased RyR2 Po from 0.3±0.1 to 0.8±0.1, n=5/6, p<0.001). These data suggest that the mechanism by which PKA phosphorylation activates RyR2 channels involves dissociation of FKBP12.6 from the channel resulting in increased sensitivity to Ca²⁺-induced activation.

Heart Failure and PKA Hyperphosphorylation Produce the Same RyR2 Defects

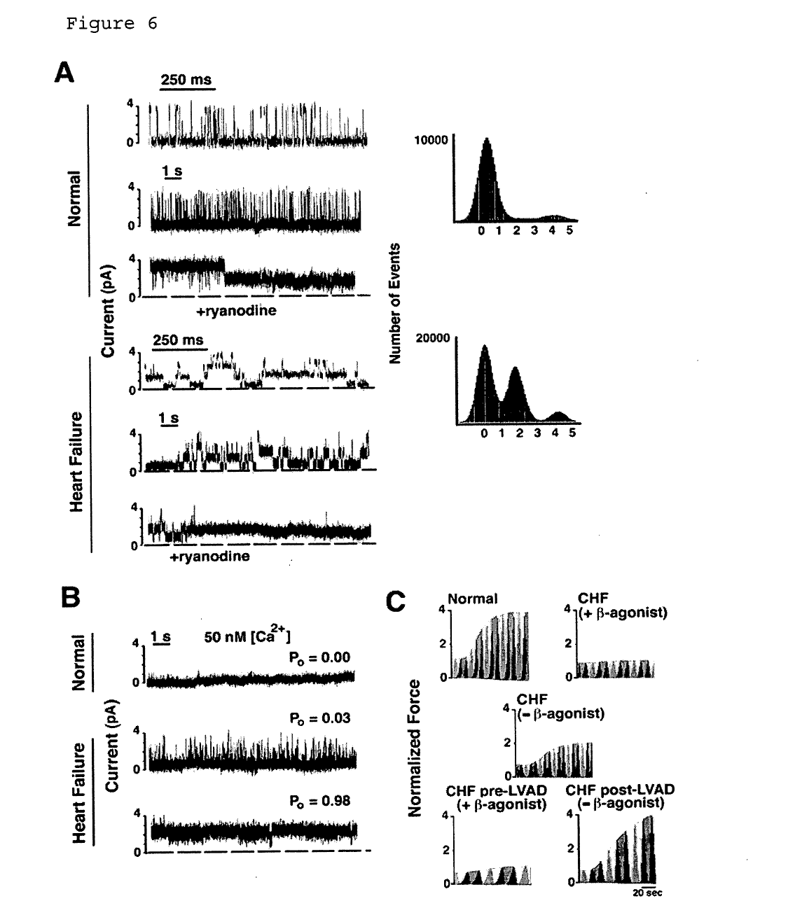

Single channel recordings of RyR2 channels from human hearts (n=21, 13 channels from 3 patients with heart failure including 3 channels from pre-LVAD treatment heart samples, 4 channels from non-failing hearts, and 4 channels from hearts post-LVAD treatment) and canine hearts (n=27, 14 channels from 2 dogs with pacing-induced heart failure, and 13 channels from non-failing hearts) revealed that the RyR2 channels from failing hearts exhibited the same alterations in single channel properties (FIGS. 6A and 6B) as the PKA phosphorylated channels (FIG. 5B). RyR2 channels from failing hearts exhibited an increased Pₒ at low cis (cytosolic) Ca²⁺ concentration (50 nM, 0.24±0.21 versus 0.002±0.001, n=27 failing hearts, n=21 non-failing and post-LVAD hearts, p<0.0001). At 50 nM cis (cytosolic) [Ca²⁺], 70% of the RyR2 channels from failing hearts (19/27) exhibited increased Pₒ(Pₒ>0) compared with 9.5% (2/21) channels from non-failing hearts. Moreover, there were two types of behavior exhibited by RyR2 channels that were active at 50 nM cis (cytosolic) [Ca²⁺]. Fifty-six % of RyR2 channels from failing hearts exhibited low levels of activity (n=15/27, Pₒ˜0.03), which is abnormal as channels from non-failing hearts are almost always completely inactive at 50 nM cis (cytosolic) [Ca²⁺] (FIG. 6B). Strikingly, 15% of the RyR2 channels from failing hearts (n=4/27) exhibited a second type of behavior that was never observed in channels from normal hearts: a long lasting subconductance state at 50 nM cis (cytosolic) [Ca²⁺] with Pₒ≈1.0 (FIG. 6B) similar to those observed following PKA phosphorylation of RyR2 channels in the bilayer (e.g., see FIG. 5B). These subconductance states had markedly increased open times (τₒ=802.1±66.7 ms) compared to the RyR2 channels from non-failing hearts (τₒ=2.2±0.7 ms) (e.g. see FIG. 6B). RyR2 channels that are active at 50 nM cis (cytosolic) [Ca²⁺] would be expected to be open throughout the cardiac cycle (both in systole and diastole).

In 52% of RyR2 channels from failing hearts subconductance states were observed (n=14/27) that were present in less than 5% of channels from normal hearts (n=1/21, p<0.001) (e.g., see FIG. 6A). The subconductance states are similar to those observed when RyR1 channels are expressed without FKBP12 (Brillantes et al., 1994) or when FKBP12.6 is dissociated from native RyR2 channels (Kaftan et al., 1996) and in PKA phosphorylated channels (FIG. 5A). As noted above channels from failing hearts also exhibited increased PKA phosphorylation (FIGS. 2A and 2B) and reduced FKBP12.6 binding (FIG. 4B). These data suggest that increased PKA phosphorylation of RyR2 in failing hearts results in dissociation of FKBP12.6 which causes defects in the single channel properties characterized by subconductance states and increased Pₒ consistent with destabilized channels and altered Ca²⁺ sensitivity (Brillantes et al., 1994).

β-Adrenergic Agonist Response Restored by LVAD Treatment

Physiologic levels of PKA phosphorylation of RyR2 would increase SR Ca²⁺ release resulting in increased cardiac muscle contractility that explains, at least in part, the inotropic effects of β-adrenergic agonists. The blunting of the β-adrenergic agonist-induced increase in cardiac contractility in failing hearts has been attributed to the downregulation and desensitization of β-adrenergic receptors in failing hearts (Bristow et al., 1992).

The blunted response to β-adrenergic agonists may in part be explained by the fact that in failing heart muscle RyR2 channels are already hyperphosphorylated (FIGS. 2A and 2B) and further PKA phosphorylation of RyR2 cannot occur. Interventions that decrease PKA phosphorylation of RyR2 back towards the levels observed in non-failing hearts should restore β-adrenergic agonist induced increases in cardiac contractility. To test this hypothesis we used muscle strips isolated from pre- and post-LVAD hearts placed in organ baths under conditions such that isoproterenol-induced contraction could be determined. Compared to normal hearts, the pre-LVAD (failing) muscle strips exhibited a blunted response to isoproterenol (FIG. 6C) which was significantly restored following LVAD treatment. LVAD treatment restores PKA phosphorylation of RyR2 to normal levels (FIGS. 2A and 2B) and reverses the defect in single channel properties of RyR2/Ca²⁺ release channels (data not shown). These data suggest that restoration of sensitivity to β-adrenergic agonists observed in the post-LVAD muscle may be explained in part by the increased availability of these RyR2 channels to be physiologically PKA phosphorylated.

Discussion

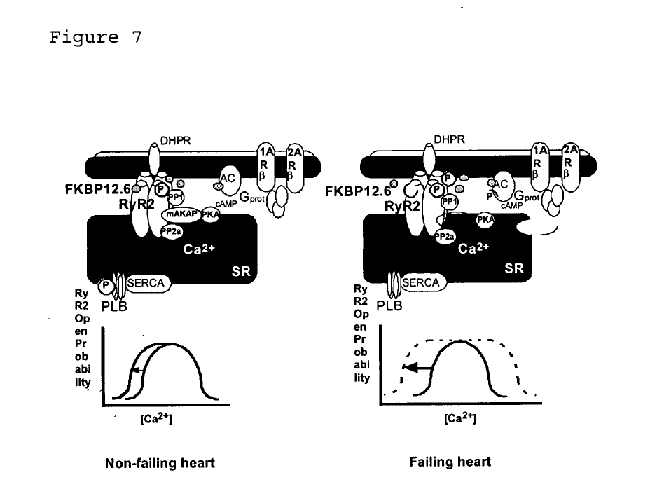

The present application discloses that PKA phosphorylation regulates FKBP12.6 binding to RyR2 providing a mechanism for modulating the sarcoplasmic reticulum Ca²⁺ release channel required for excitation-contraction coupling. PKA hyperphosphorylation of RyR2 in failing hearts resulted in the following abnormal single channel properties: 1) increased Ca²⁺ sensitivity for activation; and 2) elevated channel activity (Pₒ) associated with destabilization of the tetrameric channel complex (manifested as subconductance states including long lasting partially open states never observed in channels from non-failing hearts). Co-sedimentation and co-immunoprecipitation studies were used to define an RyR2 channel macromolecular complex that includes FKBP12.6, PKA, RII, PP1 and PP2A, and mAKAP suggesting that phosphorylation of the channel is locally controlled (FIG. 7).

FKBP12.6 and FKBP12 are integral components of the cardiac muscle RyR2 and skeletal muscle RyR1 SR Ca²⁺ release channels, respectively (Jayaraman et al., 1992; Marks, 1996) and are required for normal channel gating (Brillantes et al., 1994; Kaftan et al., 1996; Marx et al., 1998). Dissociation of FKBP12/12.6 from RyR1 or RyR2 results in three defects in channel function: 1) subconductance states with conductances equal to ¼, ½ and ¾ of the fully open channel; 2) increased Pₒ; and 3) increased sensitivity to Ca²⁺-dependent activation (Brillantes et al., 1994; Kaftan et al., 1996; Marx et al., 1998). The increased Pₒ exhibited in channels following removal of FKBP12/12.6 is explained by the increased sensitivity to Ca²⁺-dependent activation (Brillantes et al., 1994) which represents a shift to the left of the ascending portion of the bell-shaped curve describing the Ca²⁺-dependence of the RyR channels (Bezprozvanny et al., 1991). Increased Pₒ at low cytosolic Ca²⁺ (e.g., 50 nM [Ca²⁺], see FIG. 6B) would result in inappropriately active SR Ca²⁺ release channels. This would lead to depletion of SR Ca²⁺ that might impair systolic function of the heart (by diminishing the Ca²⁺ signal that activates muscle contraction). Inappropriate SR Ca²⁺ release channel activation at low cytosolic Ca²⁺ might also contribute to early and delayed after-depolarizations that trigger fatal cardiac arrhythmias and cause sudden cardiac death (Fozzard, 1992).

Alterations in RyR2 single channel function induced by PKA phosphorylation correspond to those observed when FKBP12.6 is dissociated from the channel (FIGS. 5A and B). In agreement with our findings it has been reported that PKA phosphorylation of RyR2 increases the activity of the channel (Hain et al., 1995; Valdivia et al., 1995). RyR2 channels isolated from failing hearts were PKA-hyperphosphorylated (FIGS. 2A and 2B) and exhibited the same alterations in function observed in in vitro PKA-phosphorylated channels (FIGS. 6A and 6B). Taken together these data show that PKA hyperphosphorylation of RyR2 in failing hearts causes a defect in channel function due to the dissociation of the regulatory subunit FKBP12.6. Treatment of heart failure with a mechanical device (LVAD) that improves heart function was associated with a decrease in RyR2 PKA phosphorylation to levels observed in normal human hearts (FIGS. 2A and 2B). In addition LVAD treatment resulted in normalized RyR2 single channel function (i.e., reduction in subconductance states, normalization of the Ca²⁺ sensitivity for activation and decreased Pₒ).

An additional effect of dissociation of FKBP12 from RyR1 is to uncouple gating of neighboring channels (Marx et al., 1998). We have recently found that FKBP12.6 is required for coupled gating between RyR2 channels. Coupled gating provides a mechanism whereby all of the RyR2 channels in a T-tubule/SR junction can be uniformly activated resulting in an optimal Ca²⁺ signal to trigger cardiac muscle contraction. One consequence of uncoupling RyR2 channels would be a loss of excitation-contraction coupling gain which has been observed experimentally in cardiomyopathic hearts (Gomez et al., 1997).

The present application discloses that muscle A kinase anchoring protein (mAKAP), which has been localized to cardiac SR as well as the perinuclear region (McCartney et al., 1995; Yang et al., 1998), co-sediments and co-immunoprecipitates with RyR2. mAKAP could bind directly to RyR2, similar to yotia which binds directly to the NMDA receptor (Westphal et al., 1999), or via an adaptor. The PKA regulatory subunit RII binds directly to AKAPs (Fraser and Scott, 1999) and anchors the PKA catalytic subunit. PP1 and PP2A may interact with RyR2 directly or via their own regulatory/targeting proteins possibly by binding to leucine/isoleucine zippers present in RyR2.

β-adrenergic signaling cascade components (the stimulatory G-protein Gs and adenylyl cyclase) have been localized to the transverse tubular network in rat ventricular myocytes (Laflamme and Becker, 1999). Thus, one important consequence of anchoring PKA, RII, PP1 and PP2A to the RyR2 complex and localizing upstream components of the β-adrenergic signaling cascade to the T-tubule-SR junction is that phosphorylation/dephosphorylation of RyR2 can be regulated locally at the site of excitation-contraction coupling.

The stoichiometry of PKA back-phosphorylation for the channels from failing hearts was 0.7 (compared to 3.8 for fully dephosphorylated channels and 3.1 for RyR2 from non-failing hearts) indicating that approximately three of the four PKA sites on RyR2 were phosphorylated in failing hearts compared to one or none on RyR2 from non-failing hearts. RyR2 PKA hyperphosphorylation explains the ˜60% decrease in the amount of FKBP12.6 bound to the RyR2 channels from failing hearts compared to channels from normal hearts (FIG. 4B). This decrease in FKBP12.6 binding to RyR2 channels may account for the ˜70% of RyR2 channels from failing hearts that exhibited altered single channel properties similar to those observed when FKBP12.6 is competed off from the channel using rapamycin or FK506 (Brillantes et al., 1994; Kaftan et al., 1996; Marx et al., 1998). Moreover, 15% of channels from failing hearts exhibited the most severe defect (long lasting subconductance states with Po≈1 at 50 nM cytosolic [Ca²⁺], e.g., FIG. 6B) suggesting that these channels have one or no FKBP12.6 bound.

Heart failure is the leading cause of mortality and morbidity in the United States, accounting for ˜400,000 deaths annually with ˜50% of these deaths caused by disturbances in the cardiac rhythm referred to as sudden cardiac death (SCD). A common feature of human heart failure and of many animal models of heart failure is a hyperadrenergic state, and elevated levels of circulating catecholamines are a marker for increased mortality in heart failure patients (Cohn et al., 1984).

Studies demonstrating down regulation of β-adrenergic receptors in failing heart muscle and desensitization of these receptors attributable to uncoupling from their downstream signaling molecules, G-proteins (Bristow et al., 1982), have led to some confusion since β-adrenergic blockers have proven to be one of the most important treatments for heart failure (CIBIS-II, 1999; Merit, 1999). Several studies have reported that cAMP levels and PKA activity are unchanged in failing human hearts (Kirchhefer et al., 1999; Regitz-Zagrosek et al., 1994) or that cAMP levels are reduced but PKA activity is unchanged (Bohm et al., 1994). The use of β-adrenergic blockers has been viewed as counterintuitive since the adrenergic system has been thought to be down regulated in failing hearts and drugs with negative inotropic properties are considered potentially dangerous to patients. Therefore, a mechanistic understanding of the molecular basis for the therapeutic benefit afforded by β-adrenergic blockers in patients with heart failure would be an important advance in the approach to this disease. Experiments in progress demonstrate that β-adrenergic blockers reverse the PKA hyperphosphorylation of RyR2 in dogs with heart failure induced by rapid cardiac pacing.

The present study shows that the sarcoplasmic reticulum Ca²⁺ release channel RyR2 is unexpectedly PKA-hyperphosphorylated in failing hearts. These data raise for the first time the concept that local signaling may increase rather than decrease phosphorylation of PKA substrates in cardiomyocytes from failing hearts.

One explanation for the surprising finding of PKA hyperphosphorylation of RyR2 is that targeting of phosphatases to RyR2 may be downregulated in failing hearts. Indeed, we found that the levels of PP1 and PP2A associated with RyR2 were significantly decreased in failing hearts (FIGS. 2C and D). Cellular PP1 levels are increased in failing hearts (Neumann et al., 1997); thus, the decrease in RyR2-associated PP1 must be due to a specific decrease in PP1 association with RyR2 that cannot be explained by a generalized decrease in PP1 levels in the heart.

Defects in Ca²⁺ regulation that could explain the decreased contractility observed in failing hearts, including a reduced amplitude and slowed decay of the Ca²⁺ transient, have been described (Beuckelmann et al., 1992; Morgan et al., 1990). However, the molecular basis for these defects has not been elucidated. The release and reuptake of Ca²⁺ from the sarcoplasmic reticulum controls the force of contraction during systole in the heart. SR Ca²⁺ release occurs via activation of RyR2, and Ca²⁺ reuptake occurs via the SR Ca²⁺-ATPase which in turn is regulated by phospholamban. PKA has multiple substrates in cardiomyocytes including phospholamban, the L-type Ca²⁺ channel on the sarcolemma and components of the contractile apparatus. It has been appreciated for some time that β-adrenergic agonists can modulate the activity of molecules involved in regulating cardiac contractility. Clearly, a disease as complex as heart failure involves an interplay between a number of molecules and signaling pathways that contribute to the regulation of Ca²⁺ homeostasis. One key point distinguishing the present study is the identification of a functional defect in a Ca²⁺ handling molecule that occurs not only in animal models (e.g. the paced dog model) but also in human failing hearts and is reversed by treatment of the heart failure (e.g., with an LVAD) in humans.

The present application discloses that protein kinase A (PKA) phosphorylation of the cardiac rynaodine receptor/calcium release channel (RyR2) on serine 2809 activates the channel by releasing the FK-506 binding protein 12.6 (FKBP12.6). In failing hearts (human as well as animal models of heart failure) RyR2 is PKA-hyperphosphorylated resulting in defective channels that have decreased amounts of FKBP12.6 bound to them and have increased sensitivity to calcium-induced activation. The net result of these changes is that the RyR2 channels are “leaky”. These “leaky” channels can result in depletion of intracellular stores of calcium such that there is not enough calcium in the sarcoplasmic reticulum to provide a strong stimulus for muscle contraction. This results in weak contraction of heart muscle. A second consequence of the “leaky” RyR2 channels is that they release calcium during the resting phase of the heart cycle known as diastole. This release of calcium during diastole can trigger fatal arrhythmias of the heart (e.g., ventricular tachycardia and ventricular fibrillation) that cause sudden cardiac death.

The application discloses a novel mechanism for modulating RyR2 channel function by physiologically controlling the binding of FKBP12.6 to the channel via PKA phosphorylation. Furthermore, the finding of PKA hyperphosphorylated channels with defective function in failing hearts provides a mechanism for cardiac dysfunction in heart failure. The application discloses novel targets for controlling heart muscle contraction and for treating heart failure. In addition, the application discloses methods for testing new therapeutic approaches to heart disease by assaying their effects on the RyR2 channel.

Experimental Set II

The present application provides data that demonstrate a link between altered RyR2 channel function and ventricular arrhythmias, and which suggest that defective channel function due to depletion of FKBP12.6 from the RyR2 complex may be one molecular mechanism underlying the aberrant SR Ca²⁺ release that can cause DADs and trigger ventricular arrhythmias.

The following experiments demonstrate that during exercise, PKA phosphorylation of RyR2 partially dissociates FKBP12.6 from the channel, increasing intracellular Ca²⁺ release and cardiac contractility. Data are provided to show that RyR2 channels from FKBP12.6-deficient mice and from patients with CPVT are more active during exercise compared to controls, and that cardiomyocytes from FKBP12.6-deficient mice exhibit after-depolarizations that can trigger arrhythmias that cause sudden cardiac death. New therapeutic approaches for treating cardiac arrhythmias are therefore provided, based on the finding that “leaky” RyR2 channels, induced by FKBP12.6 deficiency in the RyR2 macromolecular complex, can trigger fatal arrhythmias.

Materials and Methods

FKBP12.6-Deficient Mice

Mouse genomic λ-phage clones for the murine ortholog of the human FK506 binding protein 12.6 (FKBP12.6) were isolated from a DBA/1lacJ library using a full-length murine cDNA probe in the laboratory of Dr. Jane Bennett at Pfizer, Inc. The targeting vector was designed to delete exons 3 and 4 that contain the entire coding sequences for murine FKBP12.6 (Bennett et al., 1998) by replacing 3.5 kb of murine genomic DNA with a PGK-neo selectable marker. A 5.0 kb 5′ fragment and a 1.9 kb 3′ fragment were cloned into pJNS2, a backbone vector with PGK-neo and PGK-TK cassettes. The DBA/lacJ embryonic stem (ES) cells were grown and transfected using established protocols. Positive and negative selection was used to isolate and expand clones for identification of targeted ES cell lines. Targeted ES cells were first screened by Southern analysis on the 5′ end of the FKBP12.6 locus. A HindIII-MscI DNA fragment upstream from the FKBP12.6 5′ homology region was used to probe genomic ES cell DNA digested with BglII. This method identifies an endogenous fragment of 8.5 kb, and with correct gene targeting a 7.7 kb band is observed. Genomic Southern analysis demonstrated 5 positive targeted clones (#14, 15, 20, 34, 97) from 69 selected ES cell colonies using the upstream probe. These 5 positive ES cell lines were analyzed by PCR to confirm homologous recombination with the 3′ homology arm and the target FKBP12.6 locus. PCR primers from the PGK-neo1023F (5′-ggatgatctggacgaagagcatc-3′) and sequences 3′ in the FKBP12.6 locus, but outside of the 1.9 kb homology arm, FKBP12.6 448R (5′-ctctctgcagggggtgcattgc-3′) amplified a predicted 2.4 kb fragment in 4/5 ES cell lines (#14, 15, 20, 97) identified by 5′ Southern analysis. Karyotypic analysis of these 4 confirmed targeted ES cell lines determined that line #15 had consistent aneuploidy and was terminated. Targeted ES cells from cell line #97 were injected into C57BI/6 blastocysts. Male chimeras were bred to DBA/1lacJ females and germline offspring identified by brown coat color. Germline offspring were genotyped using 5′ Southern analysis. Positive FKBP12.6^{+/-} males and females were intercrossed and offspring resulted in FKBP12.6^{-/-} mice at approximately 25% frequency. The FKBP12.6^{-/-} mice were fertile and subsequent colonies were established by mating male and female FKBP12.6^{-/-} mice, therefore maintaining inbred genetic background. All studies performed with FKBP12.6^{-/-} mice used age and sex-matched FKBP12.6^{+/+} mice as controls. No differences were observed between FKBP12.6^{-/-} mice raised on the following backgrounds: DBA/C57BL6 mixed, pure DBA and pure C57BL6.

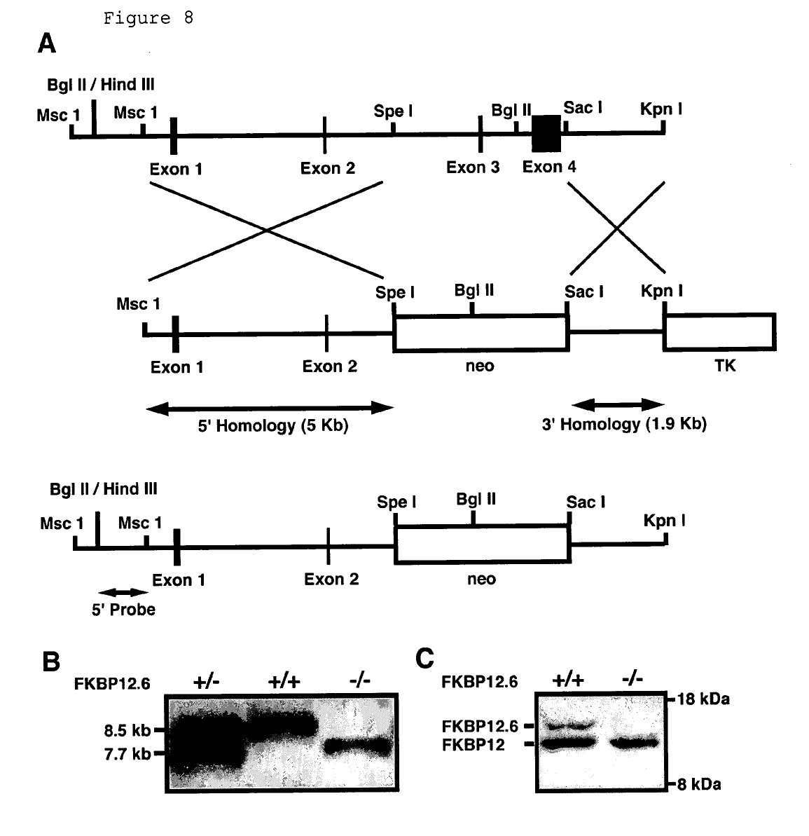

Southern blot analysis was used for identification of mouse genotypes. DNA isolated from mouse tail samples was cut with BglII and hybridized with a 5′ probe labeled with ³²P-α-dCTP using the Prime-a-Gene Labeling System (Promega). Probe DNA was obtained by isolating a 1 kb HindIII-MscI fragment of DNA located immediately upstream from the 5′ arm of the targeting vector. DNA from wild type mice and homozygous mutant mice yielded a 8.5 kb and 7.7 kb band respectively. The absence of FKBP12.6 protein was demonstrated by immunoblotting both in cardiac tissue and specifically in the RyR2 macromolecular complex by coimmunoprecipitation (FIG. 8C).

Echocardiography in Mice

Transthoracic echocardiography was performed on mice matched for age and sex using a Philips Agilent Sonos 5500 ultrasound machine with a 15 MHz transducer. Mice were anesthetized with 2.5% 2-2-2 tribromoethanol administered intra-peritoneally on a weight-adjusted basis. A short axis two-dimensional (2D) view was obtained at the level of the papillary muscle. Subsequently a 2D guided M-mode trace crossing the anterior and posterior wall was obtained at a sweep speed of 100 mm/s. 2D views were used to measure both left ventricular posterior wall thickness (LVPWT) and left ventricular end diastolic dimension (EDD). M-mode tracings were used to measure internal dimensions of both diastole and systole (LVIDd and LVIDs respectively). Fractional shortening (FS) was calculated as LVIDd−LVIDs/LVIDd.

Telemetry Recording and Exercise Testing in Mice

FKBP12.6^{+/+} and FKBP12.6^{-/-} mice were maintained and studied according to protocols approved by the Institutional Animal Care and Use Committee of Columbia University. Mice were anaesthetized using an intraperitoneal injection of ketamine (50 μg/kg) and xylazine (10 μg/kg). ECG radiotelemetry recordings of ambulatory animals were obtained >48 hours following intraperitoneal implantation (Data Sciences International, St. Paul, Minn.) (Mitchell et al., 1998). Continuous recordings were collected for each mouse but only ECG complexes with clearly defined onset and termination signals were sampled. Standard criteria were used to measure ECG parameters (Mitchell et al., 1998). QT intervals were measured to the end of the biphasic T wave (Tr+Ts; QTc=QT/(RR/100); see Mohler et al., 2003). For stress tests, mice were exercised on an inclined treadmill until exhaustion, and then intraperitoneally injected with epinephrine (0.1-0.5 mg/kg) (Mohler et al., 2003). Heart rates of ambulatory animals were determined by averaging resting heart rates over four hours.

Histological Analysis

Hearts were arrested in diastole with PBS/20 mM KCl solution and pressure fixed at 20 mmHg with 10% neutral buffered formalin. Paraffin-embedded tissues were sectioned (8 μm) and stained with hematoxylin and eosin (H&E) for light microscopy. Paraffin sections were also stained with Picrosirius red and analyzed with a polarized light microscope to evaluate the distribution of myocardial collagen. Preparation of SR From Mice

Cardiac homogenates were prepared from FKBP12.6^{+/+} and FKBP12.6^{-/-} mice by homogenizing the hearts in 1.0 ml homogenization buffer (10 mM Tris maleate, pH 6.8, 20 mM NaF, and protease inhibitors) and centrifuged at 3,000 g for 10 minutes. The supernatant was then centrifuged at 12,000 g for 20 minutes. The pellet from a final centrifugation of 120,000 g for 30 minutes was resuspended in 50 μl of resuspension buffer (10 mM Tris maleate, pH 6.8, 0.9% NaCl, 300 mM sucrose), aliquoted, and stored at −80° C. until use.

Generation and Expression of CPVT-Associated RyR2 and FKBP12.6 Mutants

Mutagenesis was performed using the Chameleon site-directed mutagenesis kit (Stratagene). The following primers were designed to introduce the indicated mutations in RyR2, and enable selection of mutant clones using introduced restriction sites: (S2246L) 5′-GTG GCT GCA GCA CTA GTG ATG GAT AAT AAT GAA CTA GC; (R2474S) 5′-G GTT TTA TTC CTT GAG AGC GTA TAC GGG ATT GAG G; (R4497C) 5′-CTA AAC TAT TTT GCA TGC AAC TTT TAC AAC ATG. The RyR2-S2809D mutation was generated using the QuikChange™ site-directed mutagenesis kit (Stratagene, La Jolla) using the following primers: 5′-C CGG ACT CGT CGT ATT GAT CAG ACA AGC CAG GTT TC, and 5′-GA AAC CTG GCT TGT CTG ATC AAT ACG ACG AGT CCG G.

The FKBP12.6 mutant D37S was generated in pRSET-A-FKBP12.6 using the reverse primer 5′-CTG TCC CGG GAT GAA CTA AAC TTC TTC CCA TTT TGG, with introduction of an XmaI site at bpl21 for mutation screening. The plasmid pCMV-FKBP12.6-D37S was generated by subcloning a BsmI-XhoI fragment from pREST-A-FKBP12.6(D37S) into the pCMV-FKBP12.6 plasmid.

HEK293 cells were co-transfected with 20 μg of RyR2 WT or mutant cDNA and with 10 μg of FKBP12.6 cDNA using the Ca²⁺ phosphate precipitation method, and vesicles containing functional RyR2 channels were prepared as previously described (Gaburjakova et al., 2001).

Immunoprecipitation of RyR2

Immunoprecipitation of RyR2 from murine heart SR was performed as described previously (Marx et al., 2000). Cardiac SR (200 μg protein) was suspended in 0.5 ml of buffer (50 mM Tris-HCl (pH 7.4), 0.9% NaCl, 0.5 mM NaF, 0.5 mM Na₃VO₄, 0.25% Triton X-100, and protease inhibitors), and incubated overnight at 4° C. with the anti-RyR-5029 antibody (immunoglobulin G (IgG) alone was used as a negative control for immunoprecipitations, data not shown). Protein A-Sepharose beads were added to the samples followed by incubation at 4° C. for 1 hour with constant mixing. Beads were washed with a 1× phosphorylation buffer [50 μM MgCl and 50 mM Tris/piperazine-N,N′-bis(2-ethanesulfonic acid), pH 6.8], and resuspended in 10 μl of a 1.5× phosphorylation buffer.

PKA Phosphorylation of RyR2

Cardiac SR membranes were prepared as described previously (Kaftan et al., 1996; Marx et al., 2000). Protein concentrations were measured by Bradford assay. Microsomes (50 μg protein) were re-suspended in 90 μl of phosphorylation buffer [50 μM MgCl and 50 mM Tris/piperazine-N,N′-bis(2-ethanesulfonic acid), pH 6.8] containing the catalytic subunit of PKA (10 units; Sigma, St. Louis, Mo.) either in the presence or absence of the specific PKA inhibitor PKI₅₋₂₄ (500 nM, Calbiochem, San Diego, Calif.). Phosphorylation was initiated with the addition of Mg-ATP (50 μM) and terminated after incubation for 30 min at room temperature by the addition of PKI. Samples were centrifuged at 100,000 g for 20 min, washed 4 times, and re-suspended in 20 μl of buffer containing 10 mM HEPES and 250 mM sucrose. Aliquots were stored at −80° C.

Back-phosphorylation of immunoprecipitated RyR2 with labeled ATP may be used to measure the extent of PKA phosphorylation of RyR2. Back-phosphorylation is initiated by adding to the immunoprecipitated RyR2 (suspended in 10 μl of 1.5× phosphorylation buffer) PKA (5 units) and Mg-ATP (33 μM) containing 10% labeled ATP, e.g., [γ-³²P]-ATP (NEN Life Sciences, Boston, Mass.). The reaction is incubated for 5 min at room temperature, and terminated by the addition of 5 μl of stop solution (4% SDS and 0.25 M DTT). Samples are heated to 95° C. and size-fractionated by 6% SDS-PAGE. Back-phosphorylation is measured by quantifying the amount of label bound to RyR2, e.g., by quantifying RyR2 radioactivity using a Molecular Dynamics Phosphorimager and ImageQuant software (Amersham Pharmacia Biotech, Piscataway, N.J.), and dividing by the amount of RyR2 protein in the immunoprecipitate as determined by immunoblotting and densitometry.

Immunoblots

Microsomes (50 μg protein) were size-fractionated by SDSPAGE (6% for RyR2, 15% for FKBP12.6) and transferred to nitrocellulose membrane overnight at 50 V. The membrane was blocked with 5% milk in TBS-Tween and incubated with primary antibody (anti-FKBP12, 1:1,000 dilution), anti-RyR (5029, 1:3,000) (Jayaraman et al., 1992), or anti-phosphoRyR2 (P2809, 1:5,000) for 1 hr at room temperature. The P2809 phosphoepitope-specific anti-RyR2 antibody is an affinity-purified, polyclonal rabbit antibody, custom-made by Zymed Laboratories (San Francisco, Calif.) using the peptide CRTRRI-(pS)-QTSQ which corresponds to RyR2, PKA-phosphorylated at S²⁸⁰⁹. After washing, the membrane was incubated with horse radish peroxidase-conjugated anti-rabbit IgG (1:5,000 dilution, Transduction Laboratories, Lexington, Ky.) for 60 min at room temperature, washed three times with Tris-buffered saline, 0.1% Tween-20, and developed using ECL enhanced chemiluminescence (Amersham Pharmacia, Piscataway, N.J.).

Cell Transfection

Human embryonic kidney (HEK) 293 cells were grown in Minimal Essential Medium (MEM) with 25 mM HEPES (Gibco), supplemented with 10% (v/v) fetal bovine serum (Gibco), penicillin (100 U/ml), streptomycin (100 mg/ml), and Lglutamine (2 mM), and transfected with 20 μg of cDNA using the Ca²⁺ phosphate precipitation method. Forty-eight hours post-transfection, SR microsomes were prepared.

Single Channel Recordings

Single-channel recordings of native RyR2 from mouse hearts or recombinant RyR2 were acquired under voltage-clamp conditions at 0 mV as described previously (Gaburjakova et al., 2001). Channel-containing vesicles were fused into a planar lipid bilayer composed from 3:1 phosphatidyl ethanolamine/phosphatidyl serine (Avanti Polar Lipids, Birmingham, Ala.) using KCl. Symmetric solutions used for channel recordings were (mmol/l): trans compartment, HEPES 250, Ba(OH)₂ 53 (in some experiments, Ba(OH)₂ was replaced by Ca(OH)₂), pH 7.35, and cis compartment, HEPES 250, Tris-base 125, EGTA 1.0, and CaCl₂ 0.5, pH 7.35. Unless otherwise indicated, single channels recordings were made in the presence of 150 nM [Ca²⁺] and 1.0 mM [Mg²⁺] in the cis compartment. At the conclusion of each experiment, ryanodine (5 mM) was applied to the cis compartment to confirm identity of the channel by specific gating block. Gating parameters (open probability Pₒ, average mean open and closed times Tₒ, T_{c}) were analyzed from digitized current recordings using Fetchan software (Axon Instruments, Union City, Calif.), and results from representative 2 min recordings are shown in the figures. RyR2 open, closed, and partial gating state probabilities were determined from all-point amplitude histograms as shown for representative examples. In a series of experiments Ca²⁺ sensitivities, as determined by incremental increases in the cis compartment from 150 nM to 10 mM, were not found significantly different between CPVT mutant and WT RyR2 channels as determined by standard regression analysis using the Hill equation. All data are expressed as mean ±SE. The unpaired Student's t-test was used for statistical comparison of mean values between experiments. A value of P<0.05 was considered statistically significant.

Action Potential Recordings

Cardiomyocytes were isolated from FKBP12.6^{+/+} or FKBP12.6^{-/-} mouse hearts using a Langendorff method (Reiken et al., 2003) and superfused with (in mM): NaCl (140), KCl (5), MgCl₂ (0.5), CaCl₂ (2.5), NaH₂PO₄ (0.33), glucose (5.5), NaF (0.5), and HEPES (5), pH 7.40, at 35-37° C. β-adrenergic stimulation of cells was produced by the addition of 1 μM isoproterenol or 1 μM epinephrine to the superfusion solution. Pipette (1-3 MΩ) filling solution was (in mM): NaCl (10), KCl (130), MgCl₂ (1.0), MgATP (5), HEPES (10), TEA-Cl (20), pH 7.20 with KOH. Axopatch 200A was used in current clamp mode to record action potentials (Mohler et al., 2003). Current injections triggered action potentials at a constant rate 12 Hz.

FKBP12.6 Binding Affinity Assay

³⁵S-labelled FKBP12.6 was made per manufacturer's protocols using the TNT® Quick Coupled Transcription/Translation system from Promega (Madison, Wis.). [³H]-ryanodine binding was performed on recombinant microsomes at 60 nM [³H]-ryanodine to insure that equal amounts of recombinant RyR2 for wild-type (WT) and each mutant was used in the assay. Microsomes (˜100 μg protein) were diluted into 100 μl of 10 mM imidazole buffer, pH 6.8, and incubated with various concentrations of [³⁵S]-FKBP12.6 at 37° C. for 60 min. The reaction was quenched by diluting the sample into 500 μl of ice-cold imidazole buffer. The samples were centrifuged at 100,000 g for 10 min, washed 3×in imidazole buffer, and the amount of bound [³⁵S]-FKBP12.6 was determined by liquid scintillation counting of the pellet.

Another method of determining the extent of binding of RyR2 and FKBP12.6 is by co-sedimentation and immunoblotting of the two proteins. Microsomes (˜200 μg protein) are suspended in 0.1 ml of imidazole buffer (5 mM imidazole, pH 7.4, 0.3 M sucrose) containing protease inhibitors, and incubated at 37° C. for 1 hr with rapamycin (Cell Signaling Technology, Beverly, Mass.). Samples are centrifuged at 95,000 g for 10 min and the supernatants are collected. Pellets are washed twice in 0.2 ml of imidazole buffer and centrifuged at 95,000 g for 10 min. The final pellet is resuspended in 0.1 ml of imidazole buffer. Both the pellet and supernatant components are fractionated by SDS-PAGE and immunoblotted for FKBP12.6 as described previously (Gaburjakova et al., 2001).

Assays for Monitoring the Effects of Test Agents on RyR2 Channel Function

PKA phosphorylation of RyR2 increases the activity of the RyR2 channel, resulting in the release of more Ca²⁺ into the cytoplasm of the cell for a given activator of the channel. Several mechanisms for suppressing activation of the RyR2 receptor by helping to maintain the channel in a closed state can be envisaged. For example, chemical agents that inhibit PKA phosphorylation of RyR2 would be expected to enhance maintenance of the closed state of the channel. Similarly, agents that inhibit the dissociation of FKBP12.6 from RyR2 and agents that mimic the binding of FKBP12.6, i.e., agents that bind to RyR2 at the FKBP12.6 binding site, but do not come off the channel when the channel is phosphorylated by PKA, would also be expected to keep the channel closed and decrease its activity in response to PKA or other activators of the RyR2 channel. These agents would all be expected to result in less Ca²⁺ leakage into the cell, and may therefore have therapeutic value in preventing DADs that can trigger cardiac arrhythmias.

An example of an agent that inhibits the dissociation of FKBP12.6 from RyR2 is JTV-519 (also known as K201) or any other compound in this class of compounds that are derivatives of 1,4-benzothiazepine) (Yano et al., 2003; Kaneko, 1994; Hachida et al., 1999; Kimura et al., 1999).

One assay for agents that may be effective in enhancing closure of a RyR2 channel, and thus in treating cardiac arrhythmias, is based on measuring the release of Ca²⁺ into cells via the RyR2 channel using calcium-sensitive fluorescent dyes (e.g., Fluo-3 or Fura-2). The assay involves loading cells with the fluorescent dye, stimulating the cells with a RyR2 activator (e.g., PKA or caffeine), and determining whether or not a chemical agent added to the cells reduces the Ca²⁺-dependent fluorescent signal (Brillantes et al., 1994; Gillo et al, 1993; Jayaraman et al., 1996). When Ca²⁺ is released into the cytoplasm of the cell, it is bound by the Ca ²⁺-sensitive dye which then emits a fluorescent signal. Ca ²⁺-dependent fluorescent signals are monitored with, for example, a photomultiplier tube or a charged coupled device (CCD) detector and analyzed with appropriate software (see Brillantes et al., 1994; Gillo et al, 1993; Jayaraman et al., 1996). This assay can be easily automated to screen large numbers of chemical agents using multiwell dishes. The assay requires the expression of RyR2 channels in a heterologous expression system such as a bacterial, yeast, insect, amphibian, plant or mammalian (including HEK293, CHO, COS-7, LM(tk⁻), mouse embryonic fibroblast NIH-3T3, or HeLa) cells (Brillantes et al., 1994). In non-muscle cells, the RyR2 receptor is expressed on the ER and when the RyR2 channel is activated, Ca²⁺ is released from the ER into the cytoplasm of the cell.

β-ARs could also be co-expressed with the RyR2 receptor. This would permit an assessment of the effects of different chemical agents on the RyR2 activation in response to β-AR stimulation.

Another assay involves measuring the level of PKA phosphorylation of RyR2 which can be used to determine the efficacy of compounds designed to inhibit PKA phosphorylation of the RyR2 channel. This assay is based on the use of antibodies that are specific for the RyR2 channel protein (anti-RyR2 antibody). In this assay, the RyR2 channel protein is immunoprecipitated with an anti-RyR2 antibody and then back-phosphorylated with PKA and [γ-³²P]-adenosine triphosphate (ATP). Samples are then size-fractionated by SDS-PAGE. The amount of radioactive ³²P label that is transferred to the RyR2 receptor protein can be measured using a phosphorimager. In another version of the assay, an antibody that binds to the phosphorylated form of the RyR2 receptor, but not to the unphosphorylated form, is used. In this case, back-phosphorylation is not necessary.

Other assays for RyR2 function involve measuring the degree of association of FKBP12.6 with the RyR2 receptor, the occurrence of subconductance states, the Ca²⁺ sensitivity for activation, the open probability (Pₒ), the open dwell time constant (τ) or the gating frequency of the channels.

FK506 and rapamycin both dissociate FKBP12.6 from RyR2. Another assay involves using FK506-Sepharose or rapamycin-Sepharose columns to screen libraries of chemical agents to identify ones that bind to the column. Binding can be assessed by thoroughly washing the columns with binding buffer followed by elution with high salt buffer. Agents that bind to the columns can be tested for their ability to bind to RyR2 (in cardiac SR or heterologous cell ER membrane preparations) and displace FKBP12.6 bound to the mutant channel. In this competition assay, the RyR2 channel is incubated with the chemical agent and then centrifuged, followed by immunoblotting the pellet versus the supernatant fractions. Agents that bind to the channel and compete off FKBP12.6 would cause FKBP12.6 to be detected in the supernatant. This could be assayed using 96-well plates with a dot-blot apparatus and immunoblotting with anti-FKBP12.6 antibody.

Chemical agents identified in these assays could be tested for their ability to inhibit isoproterenol-induced, intracellular Ca²⁺ release in cells loaded with calciumsensitive fluorescent dyes (e.g., fluo-3 or fura-2).

Chemical agents that inhibit the dissociation of FKBP12.6 binding protein from a mutant RyR2 can be identified by high throughput enzyme-linked immunosorbent assay (ELISA) that detects FKBP12.6 released into the supernatant in, for example, 96-well dishes. Following the dissociation of FKBP12.6 from the mutant RyR2, for example by addition of rapamycin or FK506 or by PKA phosphorylation of the mutant RyR2 with cAMP and ATP, an anti-FKBP12.6 antibody would be used in the ELISA. Agents that prevent release of FKBP12.6 into the supernatant would be lead candidates for novel therapeutics that could then be tested in subjects with cardiac arrhythmias.

Results

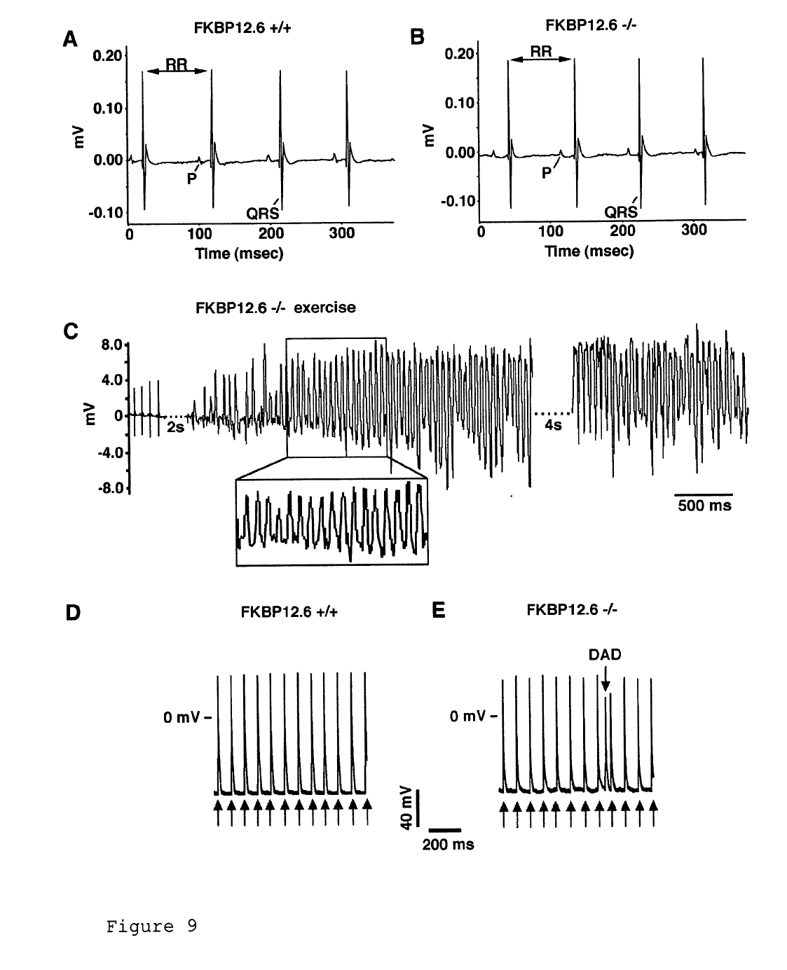

Exercise-Induced Arrhythmias in FKBP12.6-Deficient Mice FKBP12.6^{-/-} mice were generated by homologous recombination resulting in the absence of FKBP12.6 protein in the heart (FIG. 8). The hearts of male and female FKBP12.6^{-/-} mice were structurally normal based on echocardiography and histology (including absence of fibrosis, data not shown). Examination of ECG parameters of conscious FKBP12.6^{+/+} and FKBP12.6^{-/-} mice revealed no significant differences in RR (+/+, 97.3±5 msec, −/−, 92.1±4 msec), PR (+/+, 36.2±2.1 msec, −/−, 35.4±2.0 msec), QRS (+/+.17.4±1.8 msec, −/−, 16.4±1.4 msec), or rate-corrected QT intervals (QTc; +/+, 56.7±3.0 msec, −/−, 55.1±4.0 msec; n=10 +/+; 6 −/−, N.S.). Thus, FKBP12.6^{-/-} mice show no structural cardiac abnormalities, and no ECG abnormalities or arrhythmias at rest (FIGS. 9A and B).

To test for cardiac arrhythmias, FKBP12.6^{+/+} and FKBP12.6^{-/-} mice were subjected to an exercise protocol that has been used previously in mice (Mohler et al., 2003). While no FKBP12.6^{+/+} mice (0 of 10) displayed arrhythmias or syncopic events during the protocol, two of eight FKBP12.6^{-/-} mice became unresponsive for 2-5 seconds after strenuous exercise, and all of the FKBP12.6^{-/-} mice (8/8) died following exercise plus epinephrine injection. Following exercise, when epinephrine was administered, no wild type (WT) mice had arrhythmias with this stress protocol, whereas 100% (8/8) of the FKBP12.6^{-/-} mice had fatal arrhythmias consisting of a progression from a sinus rhythm (heart rate ˜700-850 bpm) with episodes of polymorphic ventricular arrhythmias (heart rate >1,200 bpm) to sustained ventricular arrhythmias (FIG. 9C). Before death, two of the mice displayed significant prolonged polymorphic arrhythmias (>6 sec), four displayed multiple short (0.5-2 s) runs of ventricular arrhythmia, and two mice died without ECG monitoring. A previous report using a different FKBP12.6^{-/-} mouse model showed that male mice had a cardiomyopathy, and in agreement with our findings no cardiac arrhythmias were observed in non-exercised animals (Xin et al., 2002). The effects of exercise were not reported in that study. The hearts from our the FKBP12.6^{-/-} mice in the present study were examined but no findings were observed to suggest a cardiomyopathy, nor were there differences between male and female mice with regard to any of the functional parameters that were measured including cardiac echocardiography, histology (data not shown) and susceptibility to ventricular arrhythmias.

Delayed After-Depolarizations in FKBPl2.6-Deficient Cardiomyocytes

To determine whether or not FKBP12.6 deficiency is associated with increased risk of delayed after-depolarizations (DADs) that can trigger arrhythmias, cardiomyocytes isolated from FKBP12.6^{-/-} and control FKBP12.6^{+/+} mice were examined with patch clamp. There were no significant changes of the single cell action potential (AP) in myocytes from the hearts of both genotypes. However, after-depolarizations were observed in FKBP12.6^{-/-} cardiomyocytes from four FKBP12.6^{-/-} mice examined under conditions that simulate exercise (i.e., following the application of isoproterenol (1 μM) or epinephrine (1 μM), and stimulation with action potentials at 12 Hz to correspond to the heart rate of ˜700-750 bpm at which fatal exercise-induced cardiac arrhythmias occurred in FKBP12.6^{-/-} mice) (FIGS. 9D and E). Since both FKBP12.6^{+/+} and FKBP12.6^{-/-} hearts were subject to increased SERCA2 (sarco-endoplasmic reticulum Ca²⁺ ATPase type 2) activity (due to PKA-dependent phosphorylation of phospholamban, data not shown), and increased L-type Ca²⁺ channel activity (due to PKA-dependent phosphorylation), and both genotypes were subject to RyR2 phosphorylation by PKA, the remaining difference is the absence of FKBP12.6 in the FKBP12.6^{-/-} mouse heart cells.

RyR2 Channels From FKBP12.6^{-/-} Mice Exhibit Defective Gating During Exercise

Native RyR2 is a tetramer comprising four RyR2 monomers, each of which binds a single FKBP12.6 molecule. FKBP12.6 stabilizes the RyR2 channel in the closed state and reduces its activity (Brillantes et al., 1994; Kaftan et al., 1996). Stimulation of the sympathetic nervous system during exercise causes the release of catecholamines that activate β-AR, which raises cAMP levels and activates protein kinase A in cardiac muscle. PKA phosphorylation of RyR2-Ser²⁸⁰⁹ dissociates FKBP12.6 from the channel complex (Marx et al., 2000) and increases the sensitivity of RyR2 to activation by [Ca²⁺] (Valdivia, 1995). Therefore, PKA phosphorylation-induced dissociation of FKBP12.6 from RyR2 and the increased sensitivity of RyR2 to [Ca²⁺] (see below) are physiological mechanisms involved in upregulating RyR2 activity (Marx et al., 2000) and increasing release of SR Ca²⁺. This occurs as part of a signaling system (the “fight or flight” stress response) that increases cardiac output to meet the metabolic demands of exercise.

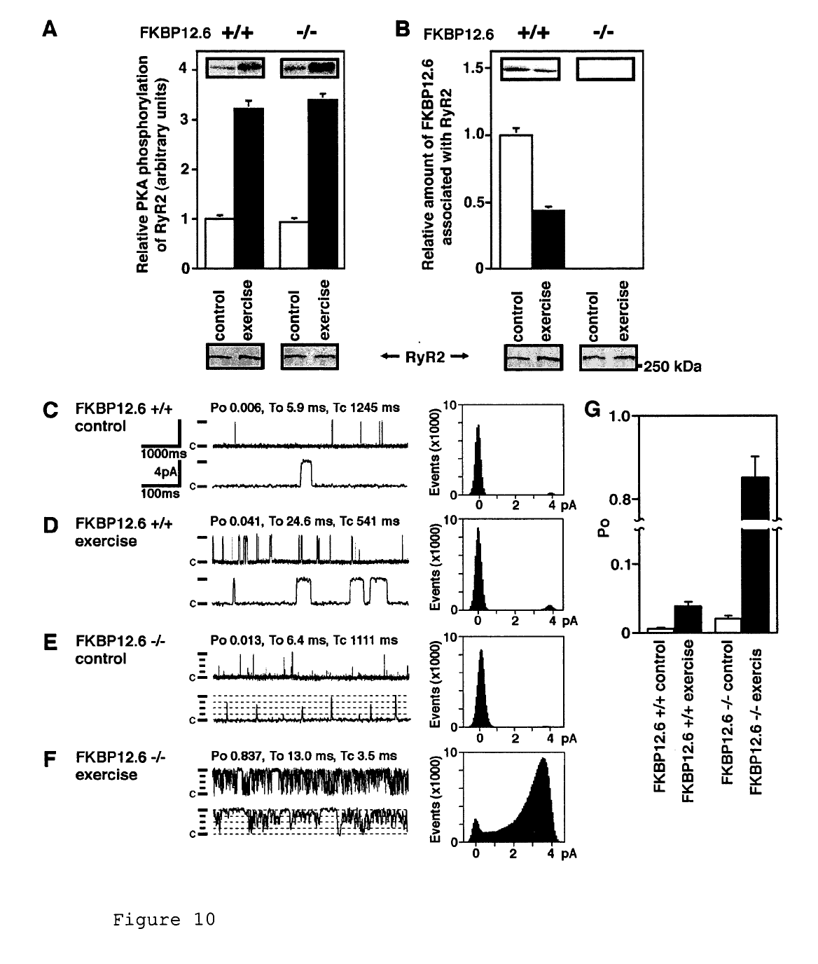

To determine the effects of exercise on RyR2, PKA phosphorylation of the channel was examined in FKBP12.6^{+/+} and FKBP12.6^{-/-} mice after exercise. RyR2s were found to be PKA-phosphorylated (FIG. 10A) and partially depleted of FKBP12.6 (FIG. 10B). RyR2s from both FKBP12.6^{+/+} and FKBP12.6^{-/-} were PKA-phosphorylated to a similar extent during exercise (FIG. 10A). During exercise the open probabilities of RyR2 channels were increased compared to channels from nonexercised animals (FIGS. 10C-F). However, FKBP12.6^{-/-} RyR2 channels from exercised mice exhibited ˜10-fold greater increase in open probability (cf. FIGS. 10D and 10F) compared to age and sex-matched FKBP12.6^{+/+} control animals subjected to the same degree of exercise (FIG. 10G). These channels were studied under conditions of low cis (cytosolic) [Ca²⁺] of 150 nM to approximate the conditions in the heart during diastole when the RyR2 channel should have very low open probability to prevent diastolic SR Ca²⁺ leak that can trigger cardiac arrhythmias. Thus, the significant increase in RyR2 open probability under these conditions in the FKBP12.6^{-/-} channels during exercise suggests that these channels could be “leaky” during diastole when the mice are exercised.

Exercise-Induced Sudden Cardiac Death is Linked to Defective RyR2 Gating

The clinical phenotype of CPVT consists of ventricular arrhythmias inducible with exercise stress testing. During exercise, patients may display a typical progression from isolated premature ventricular contractions to polymorphic ventricular tachycardia that may degenerate into ventricular fibrillation and cause sudden cardiac death (Leenhardt et al., 1995; Priori et al., 2002).

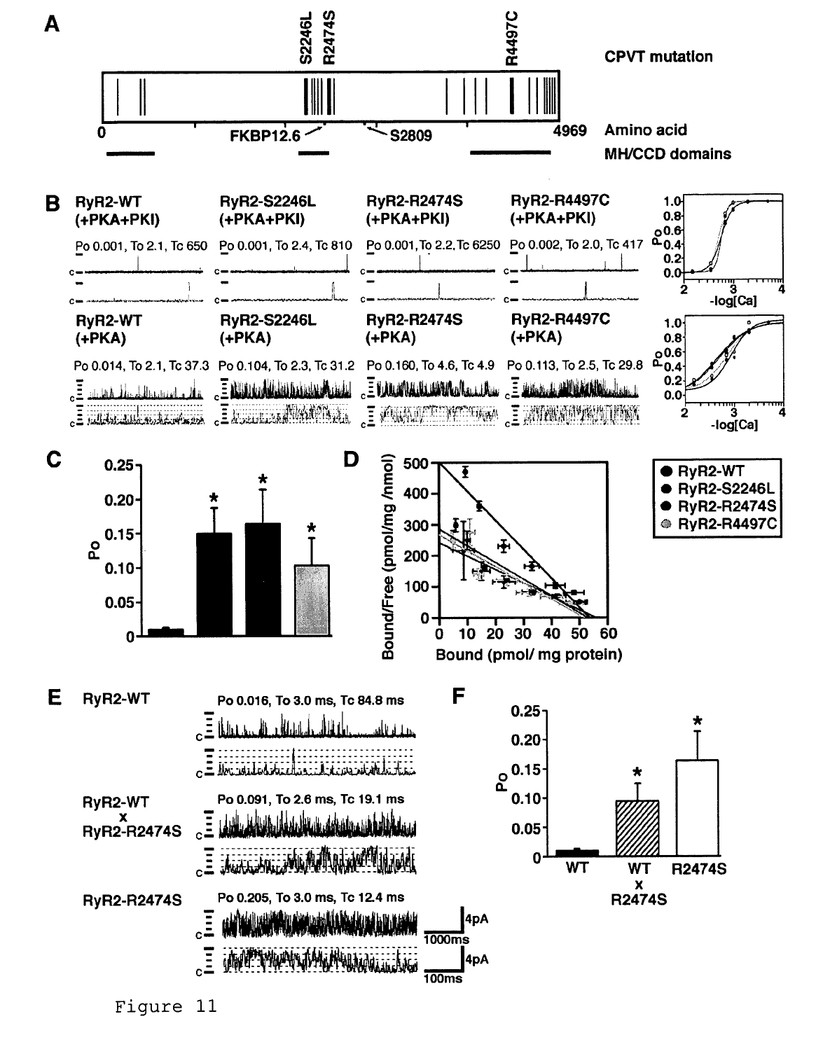

To determine whether the exercise-induced arrhythmias in CPVT patients are associated with defects in SR Ca²⁺ release channel function, three mutant forms of RyR2 corresponding to known CVPT missense mutations (RyR2-S2246L, RyR2-R2474S, and RyR2-R4497C) (FIG. 11A) were expressed. Experiments were performed at a low cis (cytosolic) [Ca²⁺] of 150 nM to approximate the conditions of cardiac muscle during diastole when the heart is relaxed and RyR2 channels have very low open probability in order to prevent SR Ca²⁺ leak. In the absence of PKA phosphorylation, WT and all three mutant RyR2 had extremely low activity (see upper tracings in FIG. 11B). There were no differences in single channel properties of WT RyR2 and mutant RyR2 examined over a wide range of cis (cytosolic) [Ca²⁺] from 50 nM to 5 mM (FIG. 11B, top right graph).

The findings that under basal conditions all three CPVT-associated mutant RyR2 exhibited normal single channel properties, indistinguishable from those of RyR2-WT channels, were not surprising given that patients with CPVT do not have arrhythmias at rest (Leenhardt et al., 1995; Priori et al., 2002). However, patients with CPVT have exercise-induced arrhythmias (Leenhardt et al., 1995; Priori et al., 2002). To approximate the effects of exercise, which activates PKA through β-AR signaling pathways in cardiomyocytes, the single channel properties of PKA-phosphorylated WT and mutant RyR2 channels were compared in planar lipid bilayers.

PKA phosphorylation significantly increased the activities (open probability; Pₒ) of the WT RyR2 and CPVT mutant channels (bottom tracings in FIG. 11B). The open probabilities of the mutant RyR2 (RyR2-S2246L, RyR2-R2474S, RyR2-R4497C) were, however, significantly higher than those for WT RyR2 channels (FIG. 11C, n=9, P<0.05). Similarly, gating frequencies were significantly higher in the mutant channels (n=9, P<0.05, data not shown). The increased open probabilities and gating frequencies exhibited by the mutant channels indicate that they are more sensitive to activation by PKA. After PKA phosphorylation both WT and CPVT-associated mutant RyR2 were more sensitive to Ca²⁺ induced activation at moderate [Ca²+]ᵢ (FIG. 11B, right), and this effect was exaggerated in the mutant channels suggesting that they are more active than the WT channels during exercise because they exhibit increased Ca²⁺-dependent activation.

To determine the effects of the CPVT mutations on FKBP12.6 binding to RyR2, microsomes were prepared from HEK293 cells expressing WT RyR2 and three CVPT mutations (RyR2-S2246L, RyR2-R2474S, and RyR2-R4497C). The amount of RyR2 in these microsomes was determined by [³H]-ryanodine binding. FKBP12.6 binding curves were obtained by incubating the microsomes with ³⁵S-labeled FKBP12.6. The dissociation constants (K_{d}) for FKBP12.6 binding to the channels (n=3) was determined from Scatchard analyses (FIG. 4D): 108.3±9.1 nM for WT RyR2, 182.7±8.1 nM for RyR2-S2246L, 215.7±6.0 nM for RyR2-R2474S, and 202.2±11.2 nM for RyR2-R4497C.

The significant increase (P<0.001) in K_{d} indicates that the CVPT mutants have decreased affinity for FKBP12.6 compared to WT channels.

Because patients with CPVT are heterozygous for the mutant RyR2 allele, the function of heterotetrameric channels was also examined by expressing equal amounts of WT RyR2 and CPVT-mutant RyR2 in HEK293 cells. Because the precise composition of any given channel studied in a bilayer experiment cannot be determined, data from multiple heterotetrameric channels were pooled. As with the homotetrameric CPVT-associated mutant RyR2 channels, heterotetrameric RyR2 channels also exhibited the same altered single channel properties, including an increase in open probability after PKA phosphorylation (FIGS. 11E and F). Taken together these data show that CPVT-associated mutant RyR2 channels exhibit significantly altered single channel properties compared to WT RyR2 channels, but only after PKA phosphorylation.

FKBP12.6 Restores Normal Gating to Defective RyR2 Channels

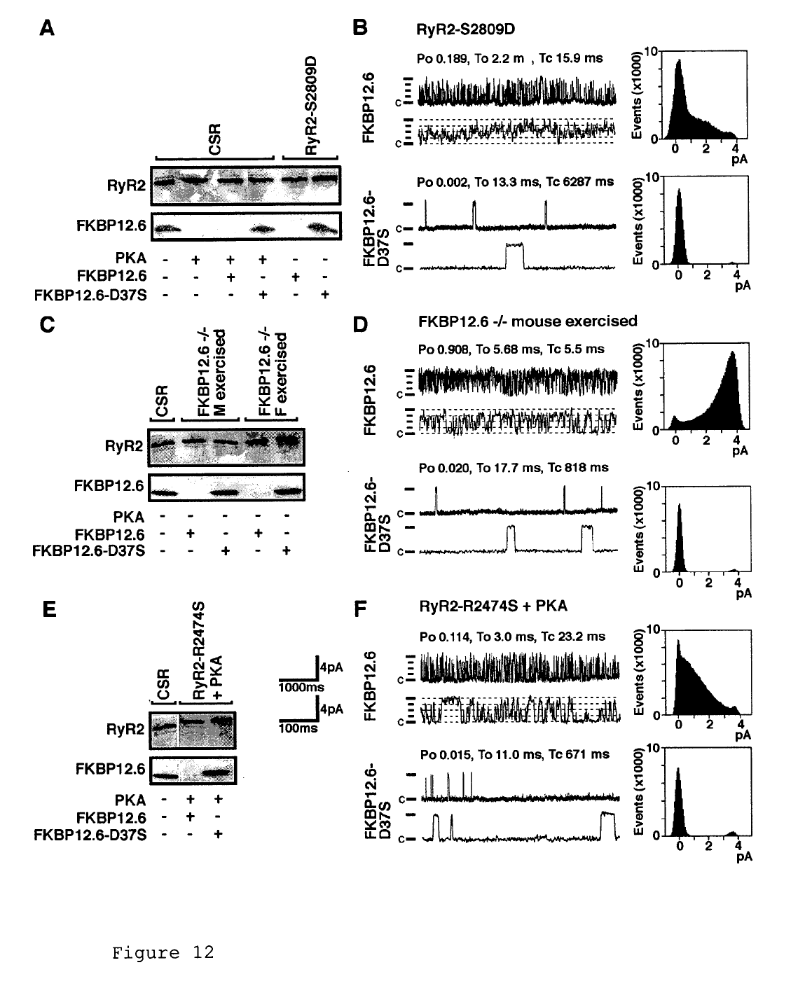

It has previously been shown that FKBP12.6 cannot bind to PKA-phosphorylated RyR2 (Marx et al., 2001; Marx et al., 2000). Results from the present study show that FKBP12.6 also cannot bind to a mutant RyR2-S2809D that mimics constitutively PKA-phosphorylated RyR2 (FIG. 12A). To determine whether or not the defective gating in the mutant CPVT-associated RyR2 was due to reduced FKBP12.6 binding, and since wild-type FKBP12.6 cannot bind to PKA phosphorylated RyR2, a mutant form of FKBP12.6 was generated in which the aspartate residue at position 37 was replaced by serine (FKBP12.6-D37S). This mutant FKBP12.6 is capable of binding to PKA-phosphorylated RyR2 and to the RyR2-S2809D mutant (FIG. 12A). The choice was made to neutralize the negative charge on Asp³⁷ because this residue is near the hydrophobic binding pocket when FKBP12 is bound to the TGFβ receptor I (Huse et al., 1999) and might be involved in PKA phosphorylation-induced dissociation of FKBP12 or FKBP12.6 from RyR1 or RyR2, respectively, when an additional negative charge is added to the channel after phosphorylation. Indeed, addition of the mutant FKBP12.6-D37S restored normal (low activity) channel function to RyR2-S2809D channels (FIG. 12B).

Moreover, in contrast to wild-type FKBP12.6, FKBP12.6-D37S was capable of binding to RyR2 channels isolated from exercised FKBP12.6^{-/-} mouse hearts (FIG. 12C) and restored normal channel function (FIG. 12D). Finally, FKBP12.6-D37S, but not wild-type FKBP12.6, also bound to PKA-phosphorylated CPVT-associated RyR2 mutant channels (FIG. 12E), and restored normal channel function (FIG. 12F). Thus, a mutant form of FKBP12.6 that, unlike wild-type FKBP12.6, binds to PKA-phosphorylated RyR2 and restores normal channel function has been generated. Taken together, these results suggest that partial depletion of FKBP12.6 from the RyR2 macromolecular complex, which occurs physiologically during exercise, is associated with increased RyR2 open probability, but more severe deficiency of FKBP12.6 in the RyR2 complex (such as in the FKBP12.6^{-/-} mouse or patients with the CPVT mutations) can result in channels with significantly increased open probability during diastole that is not observed with wild-type channels from normal hearts.

Discussion

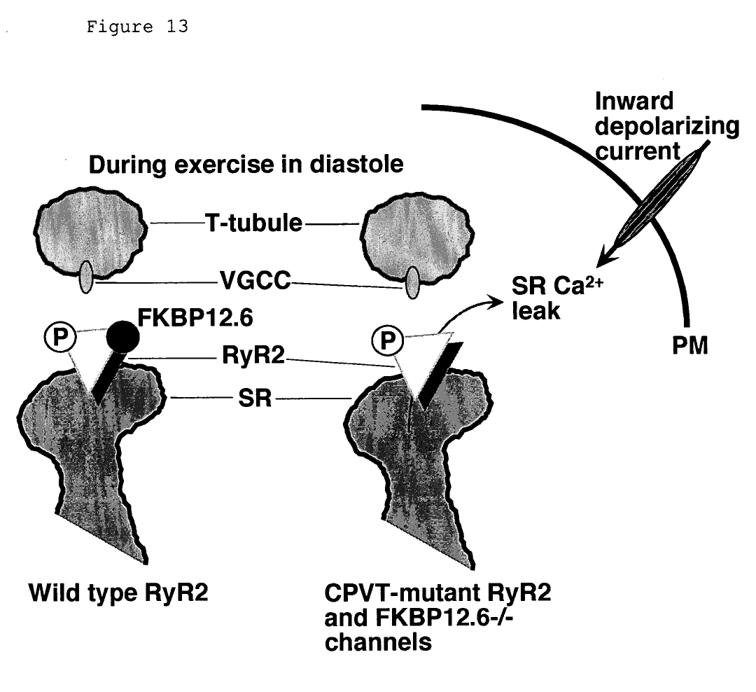

It has been shown in the present study that both RyR2 from FKBP12.6^{-/-} mice that exhibit exercise-induced sudden cardiac death, and CPVT-associated mutant RyR2s that are linked to exercise-induced arrhythmias in patients, exhibit significantly increased open probabilities under conditions that correspond to diastole in the heart during exercise. These data suggest that in vivo, SR Ca²⁺ leak through PKA-phosphorylated, FKBP12.6-depleted RyR2 can occur. Indeed, delayed after-depolarizations (DADS) were observed in cardiomyocytes from FKBP12.6^{-/-} mice during simulated exercise. Importantly, RyR2 from FKBP12.6^{-/-} mice and CPVT-RyR2 exhibit normal channel function under basal conditions, and only show defective function (i.e., increased open probability) when they are examined under conditions that correspond to exercise-induced stimulation. The increased open probabilities of the channels from FKBP12.6^{-/-} mice during exercise and the CPVT-associated mutant RyR2, combined with DADs observed in cardiomyocytes from the FKBP12.6^{-/-} mice, suggest that SR Ca²⁺ leak via RyR2 can initiate DAD-triggered arrhythmias (FIG. 13).

FKBP12.6 Deficiency Causes Exercise-Induced Sudden Cardiac Death

Cardiac ventricular arrhythmias are a major cause of mortality but the molecular bases for the triggers that initiate arrhythmias are not well understood. In the present study, it has been found that RyR2 channels from exercised FKBP12.6^{-/-} mice display dramatically increased open probabilities that were not observed in exercised FKBP12.6^{+/+} mice. These data suggest that the remaining FKBP12.6 in the RyR2 channel complexes from exercised FKBP12.6^{+/+} mice (FIG. 12B) suffice to keep PKA phosphorylated RyR2 channels closed during diastole. Previous studies examining the phenotype of an independently generated FKBP12.6^{-/-} mouse reported defects in cardiomyocyte Ca²⁺ signaling, but exercise-induced arrhythmias were not studied (Xin et al., 2002). The present results indicate that the absence of FKBP12.6 in the RyR2 channel complex predisposes mice to DADs, ventricular arrhythmias and sudden cardiac death during exercise and stimulation of the β-AR pathway.

It has previously been shown that PKA phosphorylation of RyR2 causes dissociation of FKBP12.6 from the channel complex (Marx et al., 2000). This finding was confirmed in the present study using a mutant channel, RyR2-S2809D, which mimics constitutively PKA-phosphorylated RyR2 and, therefore, cannot bind FKBP12.6 (FIGS. 12A and B). The finding that mutant FKBP12.6-D37S is able to bind PKA-phosphorylated RyR2 or RyR2-S2809D is consistent with the model that charge-repulsion, secondary to the addition of the negatively charged phosphate group to RyR2-S2809, causes dissociation of FKBP12.6 from the channel complex.

The finding that FKBP12.6-D37S could rescue the (low activity) channel phenotype in RyR2 from exercised FKBP12.6^{-/-} mice or CPVT-mutant RyR2 channels strongly suggests that the absence of FKBP12.6 following PKA phosphorylation causes RyR2 channel hyperactivity. The absence of FKBP12.6 may trigger arrhythmias in FKBP12.6^{-/-} mice and in patients with CPVT-associated RyR2 mutations. Moreover, these results suggest that increasing the binding of FKBP12.6 to phosphorylated RyR2 channels may provide a novel and very specific therapeutic strategy to prevent triggered arrhythmias in CPVT and heart failure.

PKA Phosphorylation of RyR2 is Part of the Fight-or-Flight Response

PKA phosphorylation of RyR2 occurs as part of an important physiological stress pathway known as the “fight-or-flight” response (Marks, 2000). This signaling pathway provides a mechanism whereby sympathetic nervous system activation in response to exercise or stress results in enhanced cardiac output required to meet the metabolic demands of the relevant stress. Sympathetic nervous system stimulation during exercise causes the release of catecholamines that activate β-AR, which raises intracellular cAMP levels, and hence activates PKA in cardiac muscle. PKA phosphorylation of RyR2 at Ser²⁸⁰⁹ dissociates FKBP12.6 from the channel complex, and provides a physiological mechanism, involving upregulation of RyR2 activity (Marx et al., 2000), to increase SR Ca²⁺ release in response to exercise or stress.

CPVT-Associated RyR2 Mutations Linked to Exercise-Induced Sudden Cardiac Death

Although RyR2 variants with CPVT-associated mutations exhibit reduced binding of FKBP12.6 to RyR2, these mutant RyR2 channels were able to bind FKBP12.6 under basal conditions. This finding is consistent with the fact that CPVT patients do not exhibit arrhythmias under resting conditions. Since PKA phosphorylation-induced dissociation of FKBP12.6 is part of the mechanism by which RyR2 channels are activated during exercise, the reduced affinity of FKBP12.6 likely plays a role in the increased sensitivity of mutant channels to activation by PKA. Indeed, following PKA phosphorylation, the CPVT-associated RyR2 mutations examined in this study resulted in channels that have increased activities compared to WT channels examined under the same conditions. Thus, during exercise, PKA phosphorylation of CPVT-mutant RyR2 increases the probability of after-depolarizations that can trigger arrhythmias (FIG. 13).

It was found that under basal conditions, CPVT-associated RyR2 channels exhibited normal activity, in contrast to a previous report that the RyR2-R4497C mutant had increased basal activity at very low [Ca²⁺] (Jiang et al., 2002). No difference in channel function was observed even at a [Ca²⁺] of 50 nM which is below that of resting cardiomyocytes or during diastole when cardiac muscle is relaxed. Furthermore, the fact that in the present study mutant RyR2 only exhibited defects after PKA phosphorylation, corresponding to the condition of the channels during exercise, supports the relevance of these specific alterations in channel function to the exercise-induced arrhythmias in CPVT patients. Moreover, it is difficult to understand how any defect found in the CPVT-associated RyR2 channels under non-exercise conditions can be linked to SCD in these patients because they never have arrhythmias at rest.

Delayed After-Depolarizations Associated With “Leaky” Ryanodine Receptors

DADs are oscillations in the plasma membrane potential occurring after completion of the cardiac action potential that are caused by aberrant SR Ca²⁺ release, resulting in a Ca²⁺-activated transient inward (depolarizing) current (Iₜᵢ) (Fozzard, 1992; Wit and Rosen, 1983). Previous pharmacological studies, showing that ryanodine specifically inhibits DADs, suggest that they may be due to defective SR Ca²⁺ release and implicate RyR2 in this process (Marban et al., 1986; Song and Belardinelli, 1994). The present study strengthens this mechanistic link between RyR2 and DADs in the setting of triggered ventricular arrhythmias.

DAD-induced triggered activity has been proposed as the principal mechanism for CPVT-associated, exercise-induced arrhythmias (Priori et al., 2002). The biophysical defects in CPVT-mutant channels were only observed when the channels were subjected to PKA phosphorylation, which mimics the condition of the channels during exercise. This provides further evidence supporting the relevance of these defects in channel function to the arrhythmias in patients, because the arrhythmias are also exclusively observed during stress and can be elicited with exercise testing.

In support of this mechanism, the onset of premature ventricular contractions (PVCs) in patients with CPVT during exercise testing was found to occur at a sinus rate >100 beats per minute (107±7 beats per minute, range 100-120, n=9). Ventricular tachycardia developed at a sinus rate >130 bpm (mean 148±22; range 135-204). Interestingly, the coupling interval of ventricular tachycardias (352±26 msec) was significantly shorter than that of isolated premature beats (p<0.0001), indicating that the rate dependency of arrhythmias in the carriers of the three RyR2 mutations fulfills the criteria for DAD-mediated triggered activity (Fozzard, 1992).

A Potential Molecular Mechanism For Triggered Cardiac Arrhythmias

While CPVT is a rare inherited disorder, the data showing that a defect in RyR2 function is linked to sudden cardiac death may have broader implications. The elucidation of the defect in CPVT-associated RyR2 provides a link between the phenotype of “leaky” SR Ca²⁺ release channels and cardiac arrhythmias. A transient exercise-induced defect in RyR2 function likely provides a mechanism for the exercise-induced arrhythmias in CPVT (FIG. 13).

Heart failure, a leading cause of mortality in the developed world, is associated with altered RyR2 function due to PKA hyperphosphorylation and depletion of FKBP12.6 from the channel complex that may cause aberrant SR Ca²⁺ release (Marx et al., 2000). Indeed, RyR2 from failing hearts exhibit the same defective single channel properties as the RyR2 from FKBP12.6^{-/-} mice and the CPVT-associated RyR2 examined under exercise conditions (Marx et al., 2000). This is likely because heart failure is a chronic hyperadrenergic state resulting in chronic PKA hyperphosphorylation of RyR2 channels and depletion of FKBP12.6 from the RyR2 macromolecular complex (Marx et al., 2000).

PKA-phosphorylated, CPVT-associated mutant RyR2 and WT RyR2 channels from failing human hearts both exhibit increased activities at low cytosolic [Ca²⁺] which may promote SR Ca²⁺ leak. The distinction between these two disease states is that in CPVT the defective channel function is due to inherited mutations in RyR2 that reduce the affinity for FKBP12.6 and impair the ability of the channel to close during diastole, whereas in heart failure the RyR2 channel complexes are depleted of FKBP12.6 due to chronic PKA hyperphosphorylation (Marx et al., 2000). Taken together, these data suggest that the mechanisms for triggered arrhythmias in heart failure patients may be similar to those in CPVT and in the FKBP12.6^{-/-} mouse. It should be noted that cardiac arrhythmias in failing hearts are likely due to multiple causes and those triggered by SR Ca²⁺ leak likely represent a subset of these arrhythmias.

The present studies have implications for therapeutic approaches to cardiac arrhythmias. The elucidation of the molecular mechanisms of Ca²⁺ “leak” via RyR2 in hearts of FKBP12.6^{-/-} mice and CPVT human patients has enabled the design of specific assays to identify chemical agents that counteract the leakage of Ca²⁺ by maintaining the closed state of RyR2 channels. Strategies for enhancing closure of RyR2 channels include, but are not restricted to, reducing PKA phosphorylation of the channels, inhibiting the dissociation of FKBP12.6 from the PKA-phosphorylated channel, and mimicking the binding of FKBP12.6 to the channel with an agent that is less readily dissociated by PKA phosphorylation. One of skill in the art would appreciate that a chemical agent that reduces PKA phosphorylation of a RyR2 receptor could act via multifarious mechanisms, including, inter alia, inhibiting PKA activity, or increasing the activity of endogenous phosphatases (PP1 and PP2A have been shown to be present in the RyR2 macromolecular complex; Marx et al., 2000), or increasing the activity of a phosphodiesterase which hydrolyzes cAMP (PDE4D3, which hydrolyzes cAMP, has also been shown to be present in the RyR2 macromolecular complex; Dodge et al., 2001). Leakage of Ca²⁺ may also be reduced by physically occluding transport of Ca²⁺ through the phosphorylated RyR2 channel with a chemical agent that binds directly to or is preferentially transported through the channel. The viability of at least one of the above-mentioned therapeutic strategies has already been demonstrated by the use of β-AR blocker drugs that inhibit PKA phosphorylation of RyR2 (Reiken et al., 2001) to prevent arrhythmias in patients with CPVT (Leenhardt et al., 1995).

Conclusions

Taken together, the results of the present experiments indicate that FKBP12.6 deficiency in mice and the CPVT-associated mutations in human RyR2 cause transient exercise-induced defects in channel function, suggesting that a Ca²⁺ leak through RyR2 can trigger cardiac arrhythmias. Combined with the previous set of data (Experimental Set I) showing that RyR2 is chronically depleted of FKBP12.6 in failing hearts, these findings suggest that the mechanisms underlying the ventricular arrhythmias in FKBP12.6^{-/-} mice, and in CPVT due to mutant RyR2, may be similar to those initiating ventricular arrhythmias commonly associated with heart failure. The key difference is that the channels from FKBP12.6-deficient mice and the CPVT-associated RyR2 channels only exhibit altered activity when they are transiently PKA-phosphorylated during exercise, whereas in failing hearts RyR2 channels that do not have mutations exhibit altered function on the basis of chronic PKA hyperphosphorylation (Marx et al., 2000). The elucidation of the probable molecular mechanisms of Ca²⁺ “leak” underlying ventricular arrhythmias in heart failure has enabled the design of novel therapeutic approaches targeting the RyR2 receptor.

Experimental Set III

Materials and Methods

Assaying the Effect of JTV-519 on Binding of FKBP12.6 to PKA-phosphorylated RyR2

Canine cardiac SR membranes were prepared as described previously (Kaftan et al., 1996). Ryanodine receptors (RyR2) were phosphorylated with PKA catalytic subunit (40 U; Sigma Chemical Co., St. Louis, Mo.) in the presence or absence of the PKA inhibitor PKI₅₋₂₄ in phosphorylation buffer (8 mM MgCl₂, 10 mM EGTA, and 50 mM Tris/PIPES, pH 6.8). Samples were centrifuged at 100,000×g for 10 min, washed three times in imidazole buffer (10 mM imidazole, pH 7). Recombinantly expressed FKBP12.6 (final concentration 250 nM) was added to the samples in the absence or presence of different concentrations of JTV-519. After 60 min incubation, samples were centrifuged at 100,000×g for 10 min, washed twice in imidazole buffer. Samples were heated to 95° C. and size-fractionated using SDS-PAGE. Immunoblotting of the SR microsomes was performed as described with anti-FKBP12.6 antibody (1:1,000) and anti-RyR2-5029 antibody (1:3,000) (Jayaraman et al., 1996).

Results

The results showed that JTV-519 enables FKBP12.6 to bind to PKA-phosphorylated RyR2 (partial binding at 100 nM, complete binding at 1000 nM).

### REFERENCES

Bennett, J. A., Clancy, Y. C. and McNeish, J. D. (1998) Identification and characterization of the murine FK506 binding protein (FKBP) 12.6 gene. Mamm. Genome 9: 1069-1071.

Beuckelmann, D., Nabauer, M. and Erdmann, E. (1992) Intracellular calcium handling in isolated ventricular myocytes from patients with terminal heart failure. Circulation 85: 1046-1055.

Bezprozvanny, I., Watras, J. and Ehrlich, B. (1991) Bell-shaped calcium response curves of Ins(1,4,5)P3- and calcium-gated channels from endoplasmic reticulum of cerebellum. Nature 351: 751-754.

Bohm, M., Reiger, B., Schwinger, R. H. and Erdmann, E. (1994) cAMP concentrations, cAMP dependent protein kinase activity, and phospholamban in non-failing and failing myocardium. Cardiovasc. Res. 28: 1713-1719.

Brillantes, A. B., Ondrias, K., Scott, A., Kobrinsky, E., Ondriasova, E., Moschella, M. C., Jayaraman, T., Landers, M., Ehrlich, B. E. and Marks, A. R. (1994) Stabilization of calcium release channel (ryanodine receptor) function by FK506-binding protein. Cell 77: 513-523.

Bristow, M. R., Ginsburg, R., Minobe, W., Cubicciotti, R. S., Sageman, W. S., Lurie, K., Billingham, M. E., Harrison, D. C. and Stinson, E. B. (1982) Decreased catecholamine sensitivity and beta-adrenergic-receptor density in failing human hearts. N. Engl. J. Med. 307: 205-211.

Bristow, M. R., Minobe, W., Rasmussen, R., Larrabee, P., Skerl, L., Klein, J. W., Anderson, F. L., Murray, J., Mestroni, L., Karwande, S. V. et al. (1992) Beta-adrenergic neuroeffector abnormalities in the failing human heart are produced by local rather than systemic mechanisms. J. Clin. Invest. 89: 803-815.

Cameron, A. M., Nucifora, F. C., Jr., Fung, E. T., Livingston, D. J., Aldape, R. A., Ross, C. A. and Snyder, S. H. (1997) FKBP12 binds the inositol 1,4,5-trisphosphate receptor at leucine- proline (1400-1401) and anchors calcineurin to this FK506-like domain. J. Biol. Chem. 272: 27582-27588.

CIBIS-II (1999) The cardiac insufficiency bisoprolol study II (CIBIS-II): a randomised trial. Lancet 353: 9-13.

Cohn, J. N., Levine, T. B., Olivari, M. T., Garberg, V., Lura, D., Francis, G. S., Simon, A. B. and Rector, T. (1984) Plasma norepinephrine as a guide to prognosis in patients with chronic congestive heart failure. N. Engi. J. Med. 311: 819-823.

Dodge, K. L., Khouangsathiene, S., Kapiloff, M. S., Mouton, R., Hill, E. V., Houslay, M. D., Langeberg, L. K. and Scott, J. D. (2001) MAKAP assembles a protein kinase A/PDE4 phosphodiesterase cAMP signaling module. EMBO J. 20: 1921-1930.

Fabiato, A. (1983) Calcium-induced release of calcium from the cardiac sarcoplasmic reticulum. Am. J. Physiol. 245: C1-C14.

Fisher, J. D., Krikler, D. and Hallidie-Smith, K. A. (1999) Familial polymorphic ventricular arrhythmias: a quarter century of successful medical treatment based on serial exercise-pharmacologic testing. J. Am. Coll. Cardiol. 34: 2015-2022.

Fozzard, H. A. (1992) Afterdepolarizations and triggered activity. Basic Res. Cardiol. 87: 105-113.

Franzen, P., ten Dijke, P., Ichijo, H., Yamashita, H., Schulz, P., Heldin, C. H. and Miyazono, K. (1993) Cloning of a TGF beta type I receptor that forms a heteromeric complex with the TGF beta type II receptor. Cell 75: 681-692.

Fraser, I. D. and Scott, J. D. (1999) Modulation of ion channels: a “current” view of AKAPs. Neuron 23: 423-426.

Frazier, O. H. (1994) First use of an untethered, vented electric left ventricular assist device for long-term support. Circulation 89: 2908-2914.

Gaburjakova, M., Gaburjakova, J., Reiken, S., Huang, F., Marx, S. O., Rosemblit, N. and Marks, A. R. (2001) FKBP12 binding modulates ryanodine receptor channel gating. J. Biol. Chem. 276: 16931-16935.

Gillo, B., Ma, Y. S. and Marks, A. R. (1993) Calcium entry during induced differentiation in Murine erythroleukemia cells. Blood 81: 783-792.

Go, L. O., Moschella, M. C., Watras, J., Handa, K. K., Fyfe, B. S. and Marks, A. R. (1995) Differential regulation of two types of intracellular calcium release channels during end-stage heart failure. J. Clin. Invest. 95: 888-894.

Gomez, A. M., Valdivia, H. H., Cheng, H., Lederer, M. R., Santana, L. F., Cannell, M. B., McCune, S. A., Altschuld, R. A. and Lederer, W. J. (1997) Defective excitation-contraction coupling in experimental cardiac hypertrophy and heart failure. Science 276: 800-806.

Hachida, M., Kihara, S., Nonoyama, M. et al. (1999) Protective effect of JT-519, a new 1,4-benzothiazepine derivative, on prolonged myocardial preservation. J. Card. Surg. 14: 187-193.

Hain, J., Onoue, H., Mayrleitner, M., Fleischer, S. and Schindler, H. (1995) Phosphorylation modulates the function of the calcium release channel of sarcoplasmic reticulum from cardiac muscle. J. Biol. Chem. 270: 2074-2081.

Harnick, D. J., Jayaraman, T., Ma, Y., Mulieri, P., Go, L. O. and Marks, A. R. (1995) The human type 1 inositol 1,4,5-trisphosphate receptor from T lymphocytes. Structure, localization, and tyrosine phosphorylation. J. Biol. Chem. 270: 2833-2840.

Huse, M., Chen, Y. G., Massague, J. and Kuriyan, J. (1999) Crystal structure of the cytoplasmic domain of the type I TGF beta receptor in complex with FKBP12. Cell 96: 425-436.

Jayaraman, T., Brillantes, A. -M. B., Timerman, A. P., Erdjument-Bromage, H., Fleischer, S., Tempst, P. and Marks, A. R. (1992) FK506 binding protein associated with the calcium release channel (ryanodine receptor). J. Biol. Chem. 267: 9474-9477.

Jayaraman, T., Ondrias, K., Ondriasova, E. and Marks, A. R. (1996) Regulation of the inositol 1,4,5-trisphosphate receptor by tyrosine phosphorylation. Science 272: 1492-1494.

Jiang, D., Xiao, B., Zhang, L. and Chen, S. R. (2002) Enhanced basal activity of a cardiac Ca²⁺ release channel (ryanodine receptor) mutant associated with ventricular tachycardia and sudden death. Circ. Res. 91: 218-225.

Kaftan, E., Marks, A. R. and Ehrlich, B. E. (1996) Effects of rapamycin on ryanodine receptor/Ca⁽²⁺⁾-release channels from cardiac muscle. Circ. Res. 78: 990-997.

Kaneko, N. (1994) New 1,4-benzothiazepine derivative, K201, demonstrates cardio-protective effects against sudden cardiac cell death and intracellular calcium blocking action. Drug Dev. Res. 33: 429-438.

Kapiloff, M. S., Schillace, R. V., Westphal A. M. and Scott, J. D. (1999) MAKAP: an A-kinase anchoring protein targeted to the nuclear membrane of differentiated myocytes. J. Cell Sci. 112: 2725-2736.

Kimura, J., Kawahara, M., Sakai, E. at al. (1999) Effects of a novel cardioprotective drug, JTV-519, membrane currents of guinea pig ventricular myocytes. Jpn. J. Pharmacol. 79: 275-281.

Kirchhefer, U., Schmitz, W., Scholz, H. and Neumann, J. (1999) Activity of cAMP-dependent protein kinase and Ca2+/calmodulin-dependent protein kinase in failing and nonfailing human hearts. Cardiovasc. Res. 42: 254-261.

Laflamme, M. A. and Becker, P. L. (1999) Gs and adenylyl cyclase in transverse tubules of heart: implications for cAMP-dependent signaling. Am. J. Phys. 277: H1841-H1848.

Laitinen, P. J., Brown, K. M., Piippo, K., Swan, H., Devaney, J. M. et al. (2001) Mutations of the cardiac ryanodine receptor (RyR2) gene in familial polymorphic ventricular tachycardia. Circulation 103: 485-490.

Leenhardt, A., Lucet, V., Denjoy, I., Grau, F., Ngoc, D. D. and Coumel, P. (1995) Catecholaminergic polymorphic ventricular tachycardia in children: a 7-year follow-up of 21 patients. Circulation 91: 1512-1519.

Levin, H. R., Oz, M. C., Chen, J. M., Packer, M., Rose, E. A. and Burkhoff, D. (1995) Reversal of chronic ventricular dilation in patients with end-stage cardiomyopathy by prolonged mechanical unloading. Circulation 91: 2717-2720.

Lorenz, M. C. and Heitman, J. (1995) TOR mutations confer rapamycin resistance by preventing interaction with FKBP12-rapamycin. J. Biol. Chem. 270: 27531-27537.

MacDougall, L. K., Jones, L. R. and Cohen, P. (1991) Identification of the major protein phosphatases in mammalian cardiac muscle which dephosphorylate phospholamban. Eur. J. Biochem. 196: 725-734.

Marban, E., Robinson, S. W. and Wier, W. G. (1986) Mechanisms of arrhythmogenic delayed and early afterdepolarizations in ferret ventricular muscle. J. Clin. Invest. 78: 1185-1192.

Marks, A. R. (1996) Cellular functions of immunophilins. Physiol. Rev. 76: 631-649.

Marks, A. R. (2000) Cardiac intracellular calcium release channels: role in heart failure. Circ. Res. 87: 8-11.

Marx, S. O., Ondrias, K. and Marks, A. R. (1998) Coupled gating between individual skeletal muscle Ca²⁺ release channels (ryanodine receptors). Science 281: 818-821.

Marx, S. O., Reiken S, Hisamatsu, Y., Gaburjakova, M., Gaburjakova, J., Yang, Y. M., Rosemblit, N. and Marks, A. R. (2001) Phosphorylation-dependent regulation of ryanodine receptors: a novel role for leucine/isoleucine zippers. J. Cell. Biol. 153: 699-708.

Marx, S. O., Reiken, S., Hisamatsu, Y., Jayaraman, T., Burkhoff, D., Rosemblit, N. and Marks, A. R. (2000) PKA phosphorylation dissociates FKBP12.6 from the calcium release channel (ryanodine receptor): defective regulation in failing hearts. Cell 101: 365-376.

McCartney, S., Little, B. M., Langeberg, L. K. and Scott, J. D. (1995) Cloning and characterization of A-kinase anchor protein 100 (AKAP100). A protein that targets A-kinase to the sarcoplasmic reticulum. J. Biol. Chem. 270: 9327-9333.

Merit, H. F. (1999) Effect of metoprolol CR/XL in chronic heart failure: metoprolol CR/XL randomised intervention trial in congestive heart failure (MERIT-HF). Lancet 353: 2001-2007.

Mitchell, G. F., Jeron, A. and Koren, G. (1998) Measurement of heart rate and Q-T interval in the conscious mouse. Am. J. Physiol. 274: H747-H751.

Mohler, P. J., Schott, J. J., Gramolini, A. O., Dilly, K. W., Guatimosim, S. et al. (2003) Ankyrin-B mutation causes type 4 long-QT cardiac arrhythmia and sudden cardiac death. Nature 421: 634-9.

Morgan, J., Erny, R., Allen, P., Grossman, W. and Gwathmey, J. (1990) Abnormal intracellular calcium handling: a major cause of systolic and diastolic dysfunction in ventricular myocardium from patients with end-stage heart failure. Circulation 81(suppl III): III21-III32.

Moschella, M. C. and Marks, A. R. (1993) Inositol 1,4,5-trisphosphate receptor expression in cardiac myocytes. J. Cell. Biol. 120: 1137-1146.

Nabauer, M., Callewart, G., Cleeman, L. and Morad, M. (1989) Regulation of calcium release is gated by calcium current, not gating charge, in cardiac myocytes. Science 244: 800-803.

Neumann, J., Eschenhagen, T., Jones, L. R., Linck, B., Schmitz, W., Scholz, H. and Zimmermann, N. (1997) Increased expression of cardiac phosphatases in patients with end-stage heart failure. J. Mol. Cell. Cardiol. 29: 265-272.

Otsu, K., Willard, H. F., Khanna, V. K., Zorato, F., Green, N. M. and MacLennan, D. H. (1990) Molecular cloning of cDNA encoding the Ca-2+release channel (ryanodine receptor) of rabbit cardiac muscle sarcoplasmic reticulum. J. Biol. Chem. 265: 13472-13483.

Pogwizd, S. M., McKenzie, J. P. and Cain, M. E. (1998) Mechanisms underlying spontaneous and induced ventricular arrhythmias in patients with idiopathic dilated cardiomyopathy. Circulation 98: 2404-2414.

Pogwizd, S. M., Schlotthauer, K., Li, L., Yuan, W. and Bers, D. M. (2001) Arrhythmogenesis and contractile dysfunction in heart failure: Roles of sodium-calcium exchange, inward rectifier potassium current, and residual beta-adrenergic responsiveness. Circ. Res. 88: 1159-1167.

Priori, S. G., Napolitano, C., Memmi, M., Colombi, B., Drago, F. et al. (2002) Clinical and molecular characterization of patients with catecholaminergic polymorphic ventricular tachycardia. Circulation 106: 69-74.

Priori, S. G., Napolitano, C., Tiso, N., Memmi, M., Vignati, G., Bloise, R., Sorrentino, V. V. and Danieli, G. A. (2001) Mutations in the cardiac ryanodine receptor gene (hRyR2) underlie catecholaminergic polymorphic ventricular tachycardia. Circulation 103: 196-200.

Regitz-Zagrosek, V., Hertrampf, R., Steffen, C., Hildebrandt, A. and Fleck, E. (1994) Myocardial cyclic AMP and norepinephrine content in human heart failure. Eur. Heart J. 15 Suppl. D: 7-13.

Reiken, S., Gaburjakova, M., Gaburjakova, J., He, Kl Kl., Prieto, A. et al. (2001) Beta-adrenergic receptor blockers restore cardiac calcium release channel (ryanodine receptor) structure and function in heart failure. Circulation 104: 2843-2848.

Reiken, S., Gaburjakova, M., Guatimosim, S., Gomez, A. M., D'Armiento, J. et al. (2003) Protein kinase A phosphorylation of the cardiac calcium release channel (ryanodine receptor) in normal and failing hearts. Role of phosphatases and response to isoproterenol. J. Biol. Chem. 278: 444-453.

Song, Y. and Belardinelli, L. (1994) ATP promotes development of afterdepolarizations and triggered activity in cardiac myocytes. Am. J. Physiol. 267: H2005-2011.

Swan, H., Piippo, K., Viitasalo, M., Heikkila, P., Paavonen, T. et al. (1999) Arrhythmic disorder mapped to chromosome 1q42-q43 causes malignant polymorphic ventricular tachycardia in structurally normal hearts. J. Am. Coll. Cardiol. 34: 2035-42.

Takeshima, H., Nishimura, S., Matsumoto, T., Ishido, H., Kangawa, K., Minamino, N., Matsuo, H., Ueda, M., Hanaoka, H., Hirose, T. and Numa, S. (1989) Primary structure and expression from complementary DNA of skeletal muscle ryanodine receptor. Nature 339: 439-445.

Valdivia, H. H., Kaplan, J. H., Ellis-Davies, G. C. and Lederer, W. J. (1995) Rapid adaptation of cardiac ryanodine receptors: modulation by Mg²+ and phosphorylation. Science 267: 1997-2000.

Wang, J., Yi, G. H., Knecht, M., Cai, B. L., Poposkis, S., Packer, M. and Burkhoff, D. (1997) Physical training alters the pathogenesis of pacing-induced heart failure through endothelium-mediated mechanisms in awake dogs. Circulation 96: 2683-2692.

Westphal, R. S., Tavalin, S. J., Lin, J. W., Alto, N. M., Fraser, I. D., Langeberg, L. K., Sheng, M. and Scott, J. D. (1999) Regulation of NMDA receptors by an associated phosphatase-kinase signaling complex. Science 285: 93-96.

Wit, A. L. and Rosen, M. R. (1983) Pathophysiologic mechanisms of cardiac arrhythmias. Am. Heart J. 106: 798-811.

Xin, H. B., Senbonmatsu, T., Cheng, D. S., Wang, Y. X., Copello, J. A. et al. (2002) Oestrogen protects FKBP12.6 null mice from cardiac hypertrophy. Nature 416: 334-338.

Yamamoto-Hino, M., Sugiyama, T., Hikiti, K., Mattei, M. G., Hasegawa, K., Sekine, S., Sakurada, K., Miyawaki, A., Furuichi, T., Hasegawa, M. and Mikoshiba, K. (1994) Cloning and characterization of human type 2 and type 3 inositol 1,4,5-trisphosphate receptors. Recept. Channels 2: 9-22.

Yang, J., Drazba, J. A., Ferguson, D. G. and Bond, M. (1998) A-kinase anchoring protein 100 (AKAP100) is localized in multiple subcellular compartments in the adult rat heart. J. Cell Biol. 142: 511-522.

Yano, M., Kobayashi, S., Kohno, M., Doi, M., Tokuhisa, T., Okuda, S., Suetsugu, M., Hisaoka, T., Obayashi, M., Ohkusa, T., Kohno, M. and Matsuzaki, M. (2003) FKBP12.6-mediated stabilization of calcium-release channel (ryanodine receptor) as a novel therapeutic strategy against heart failure. Circulation 107: 477-484.

What is claimed is:

1. A method for treating a subject afflicted with atrial tachyarrhythmia comprising administering to the subject a therapeutically effective amount of an agent which inhibits protein kinase A (PKA) phosphorylation of a type 2 ryanodine receptor (RyR2) in the subject's heart, thereby treating the subject.

2. The method of claim 1, wherein PKA phosphorylation of the RyR2 receptor causes dissociation of a FKBP12.6 binding protein from the RyR2 receptor.

3. The method of claim 1, wherein the atrial tachyarrhythmia is an atrial fibrillation or a supraventricular tachyarrhythmia.

4. A method for treating a subject afflicted with atrial tachyarrhythmia comprising administering to the subject a therapeutically effective amount of an agent which inhibits dissociation of a FKBP12.6 binding protein from a type 2 ryanodine (RyR2) receptor in the subject's heart, thereby treating the subject.

5. The method of claim 4, wherein the atrial tachyarrhythmia is an atrial fibrillation or a supra-ventricular tachyarrhythmia.

6. The method of claim 4, wherein the agent is JTV-519.

7. A method for treating a subject afflicted with atrial tachyarrhythmia comprising administering to the subject a therapeutically effective amount of an agent which mimics binding of a FKBP12.6 binding protein to a type 2 ryanodine receptor (RyR2) of the subject's heart, thereby treating the subject.

8. The method of claim 7, wherein the atrial tachyarrhythmia is an atrial fibrillation or a supra-ventricular tachyarrhythmia.

9. An article of manufacture comprising (i) a packaging material having therein an agent which inhibits protein kinase A (PKA) phosphorylation of a type 2 ryanodine receptor (RyR2) and (ii) a label indicating a use for the agent in treating a subject afflicted with atrial tachyarrhythmia.

10. An article of manufacture comprising (i) a packaging material having therein an agent which inhibits dissociation of a FKBP12.6 binding protein from a type 2 ryanodine receptor (RyR2) and (ii) a label indicating a use for the agent in treating a subject afflicted with atrial tachyarrhythmia.

11. The article of manufacture of claim 10, wherein the agent is JTV-519.

12. An article of manufacture comprising (i) a packaging material having therein an agent which mimics binding of a FKBP12.6 binding protein to a type 2 ryanodine receptor (RyR2) and (ii) a label indicating a use for the agent in treating a subject afflicted with atrial tachyarrhythmia.

13. A method for inhibiting the onset of atrial tachyarrhythmia in a subject comprising administering to the subject a prophylactically effective amount of an agent which inhibits protein kinase A (PKA) phosphorylation of a type 2 ryanodine receptor (RyR2) in the subject's heart, thereby inhibiting the onset of an atrial tachyarrhythmia in the subject.

14. The method of claim 13, wherein PKA phosphorylation of the RyR2 receptor causes dissociation of a FKBP12.6 binding protein from the RyR2 receptor.

15. The method of claim 13, wherein the atrial tachyarrhythmia is an atrial fibrillation or a supraventricular tachyarrhythmia.

16. A method for inhibiting the onset of atrial tachyarrhythmia in a subject comprising administering to the subject a prophylactically effective amount of an agent which inhibits dissociation of a FKBP12.6 binding protein from a type 2 ryanodine (RyR2) receptor in the subject's heart, thereby inhibiting the onset of atrial tachyarrhythmia in the subject.

17. The method of claim 16, wherein the atrial tachyarrhythmia is an atrial fibrillation or a supra-ventricular tachyarrhythmia.

18. The method of claim 16, wherein the agent is JTV-519.

19. A method for inhibiting the onset of atrial tachyarrhythmia in a subject comprising administering to the subject a prophylactically effective amount of an agent which mimics binding of a FKBP12.6 binding protein to a type 2 ryanodine receptor (RyR2) of the subject's heart, thereby inhibiting the onset of atrial tachyarrhythmia in the subject.

20. The method of claim 19, wherein the atrial tachyarrhythmia is an atrial fibrillation or a supraventricular tachyarrhythmia.

21. An article of manufacture comprising (i) a packaging material having therein an agent which inhibits protein kinase A (PKA) phosphorylation of a type 2 ryanodine receptor (RyR2) and (ii) a label indicating a use for the agent in inhibiting the onset of atrial tachyarrhythmia in a subject.

22. An article of manufacture comprising (i) a packaging material having therein an agent which inhibits dissociation of a FKBP12.6 binding protein from a type 2 ryanodine receptor (RyR2) and (ii) a label indicating a use for the agent in inhibiting the onset of atrial tachyarrhythmia in a subject.

23. The article of manufacture of claim 22, wherein the agent is JTV-519.

24. An article of manufacture comprising (i) a packaging material having therein an agent which mimics binding of a FKBP12.6 binding protein to a type 2 ryanodine receptor (RyR2) and (ii) a label indicating a use for the agent in inhibiting the onset of atrial tachyarrhythmia in a subject.

25. A method for treating a subject afflicted with exercise-induced or stress-induced cardiac arrhythmia comprising administering to the subject a therapeutically effective amount of an agent which inhibits protein kinase A (PKA) phosphorylation of a type 2 ryanodine receptor (RyR2) in the subject's heart, thereby treating the subject.

26. The method of claim 25, wherein PKA phosphorylation of the RyR2 receptor causes dissociation of a FKBP12.6 binding protein from the RyR2 receptor.

27. The method of claim 25, wherein the cardiac arrhythmia is a ventricular fibrillation or a ventricular tachycardia.

28. The method of claim 27, wherein the subject is afflicted with catecholaminergic polymorphic ventricular tachycardia (CPVT).

29. A method for treating a subject afflicted with exercise-induced or stress-induced cardiac arrhythmia comprising administering to the subject a therapeutically effective amount of an agent which inhibits dissociation of a FKBP12.6 binding protein from a type 2 ryanodine (RyR2) receptor in the subject's heart, thereby treating the subject.

30. The method of claim 29, wherein the cardiac arrhythmia is a ventricular fibrillation or a ventricular tachycardia.

31. The method of claim 30, wherein the subject is afflicted with catecholaminergic polymorphic ventricular tachycardia (CPVT).

32. The method of claim 29, wherein the agent is JTV-519.

33. A method for treating a subject afflicted with exercise-induced or stress-induced cardiac arrhythmia comprising administering to the subject a therapeutically effective amount of an agent which mimics binding of a FKBP12.6 binding protein to a type 2 ryanodine receptor (RyR2) of the subject's heart, thereby treating the subject.

34. The method of claim 33, wherein the cardiac arrhythmia is a ventricular fibrillation or a ventricular tachycardia.

35. The method of claim 34, wherein the subject is afflicted with catecholaminergic polymorphic ventricular tachycardia (CPVT).

36. An article of manufacture comprising (i) a packaging material having therein an agent which inhibits protein kinase A (PKA) phosphorylation of a type 2 ryanodine receptor (RyR2) and (ii) a label indicating a use for the agent in treating a subject afflicted with exercise-induced or stress-induced cardiac arrhythmia.

37. An article of manufacture comprising (i) a packaging material having therein an agent which inhibits dissociation of a FKBP12.6 binding protein from a type 2 ryanodine receptor (RyR2) and (ii) a label indicating a use for the agent in treating a subject afflicted with exercise-induced or stress-induced cardiac arrhythmia.

38. The article of manufacture of claim 37, wherein the agent is JTV-519.

39. An article of manufacture comprising (i) a packaging material having therein an agent which mimics binding of a FKBP12.6 binding protein to a type 2 ryanodine receptor (RyR2) and (ii) a label indicating a use for the agent in treating a subject afflicted with exercise-induced or stress-induced cardiac arrhythmia.

40. A method for inhibiting the onset of exercise-induced or stress-induced cardiac arrhythmia in a subject comprising administering to the subject a prophylactically effective amount of an agent which inhibits protein kinase A (PKA) phosphorylation of a type 2 ryanodine receptor (RyR2) in the subject's heart, thereby inhibiting the onset of exercise-induced or stress-induced cardiac arrhythmia in the subject.

41. The method of claim 40, wherein PKA phosphorylation of the RyR2 receptor causes dissociation of a FKBP12.6 binding protein from the RyR2 receptor.

42. The method of claim 40, wherein the cardiac arrhythmia is a ventricular fibrillation or a ventricular tachycardia.

43. The method of claim 42, wherein the subject is afflicted with catecholaminergic polymorphic ventricular tachycardia (CPVT).

44. A method for inhibiting the onset of exercise-induced or stress-induced cardiac arrhythmia comprising administering to the subject a prophylactically effective amount of an agent which inhibits dissociation of a FKBP12.6 binding protein from a type 2 ryanodine (RyR2) receptor in the subject's heart, thereby inhibiting the onset of exercise-induced or stress-induced cardiac arrhythmia in the subject.

45. The method of claim 44, wherein the cardiac arrhythmia is a ventricular fibrillation or a ventricular tachycardia.

46. The method of claim 45, wherein the subject is afflicted with catecholaminergic polymorphic ventricular tachycardia (CPVT).

47. The method of claim 44, wherein the agent is JTV-519.

48. A method for inhibiting the onset of exercise-induced or stress-induced cardiac arrhythmia comprising administering to the subject a prophylactically effective amount of an agent which mimics binding of a FKBP12.6 binding protein to a type 2 ryanodine receptor (RyR2) of the subject's heart, thereby inhibiting the onset of exercise-induced or stress-induced cardiac arrhythmia in the subject.

49. The method of claim 48, wherein the cardiac arrhythmia is a ventricular fibrillation or a ventricular tachycardia.

50. The method of claim 49, wherein the subject is afflicted with catecholaminergic polymorphic ventricular tachycardia (CPVT).

51. An article of manufacture comprising (i) a packaging material having therein an agent which inhibits protein kinase A (PKA) phosphorylation of a type 2 ryanodine receptor (RyR2) and (ii) a label indicating a use for the agent in inhibiting the onset of exercise-induced or stress-induced cardiac arrhythmia in a subject.

52. An article of manufacture comprising (i) a packaging material having therein an agent which inhibits dissociation of a FKBP12.6 binding protein from a type 2 ryanodine receptor (RyR2) and (ii) a label indicating a use for the agent in inhibiting the onset of exercise-induced or stress-induced cardiac arrhythmia in a subject.

53. The article of manufacture of claim 52, wherein the agent is JTV-519.

54. An article of manufacture comprising (i) a packaging material having therein an agent which mimics binding of a FKBP12.6 binding protein to a type 2 ryanodine receptor (RyR2) and (ii) a label indicating a use for the agent in inhibiting the onset of exercise-induced or stress-induced cardiac arrhythmia in a subject.
